(19) United States
(12) Patent Application Publication
(10) Pub. No.: US 2026/0345512 A1
(43) Pub. Date: Nov. 5, 2026

(54) TRANSDERMAL DELIVERY OF DEXTROMETHORPHAN

(71) Applicant: SHINKEI THERAPEUTICS, INC., Princeton, NJ (US)

(72) Inventors: Suresh BORSADIA, Plainsboro, NJ (US); Kalpana PATEL, West Windsor, NJ (US); Hock S. TAN, East Brunswick, NJ (US); Krunal RAVAL, Ahmedabad, Gujarat (IN)

(21) Appl. No.: 19/117,617

(22) PCT Filed: Oct. 5, 2023
(86) PCT No.: PCT/US2023/076067

§ 371 Date: Apr. 1, 2025

Related U.S. Application Data
(60) Provisional application No. 63/413,836, filed on Oct. 6, 2022.

(51) Int. Cl. A61K 9/70; A61K 31/23; A61K 31/485

(52) U.S. Cl. CPC A61K 9/7038; A61K 31/23; A61K 31/485

(57) ABSTRACT

Provided herein are novel transdermal delivery devices (or patches) comprising dextromethorphan, pharmaceutical compositions comprising dextromethorphan, methods of preparation thereof, and methods of administering dextromethorphan transdermally. Also provided herein are methods of treating various diseases and disorders such as neurological diseases or disorders (e.g., PBA) using the transdermal delivery devices and/or pharmaceutical compositions herein.

This application claims the benefit of U.S. Provisional Application No. 63/413,836, filed Oct. 6, 2022, the entire contents of which are incorporated herein by reference.

In various embodiments, the present disclosure generally relates to transdermal delivery devices comprising dextromethorphan, methods of preparing, and uses thereof, for example, for use in treating a disease or disorder such as a neurological disease described herein.

### BACKGROUND

NUEDEXTA® (dextromethorphan hydrobromide and quinidine sulfate) capsules, 20 mg/10 mg is a combination product containing dextromethorphan hydrobromide (an uncompetitive N-methyl-D-aspartate [NMDA] receptor antagonist and sigma-1 agonist) and quinidine sulfate (a CYP450 2D6 inhibitor). This product is indicated for the treatment of pseudobulbar affect (PBA). Dextromethorphan hydrobromide is the pharmacologically active ingredient of NUEDEXTA® that acts on the central nervous system (CNS). Quinidine sulfate is a specific inhibitor of CYP2D6-dependent oxidative metabolism used in NUEDEXTA® to increase the systemic bioavailability of dextromethorphan.

The recommended starting dose of NUEDEXTA® (dextromethorphan hydrobromide and quinidine sulfate) capsules, 20 mg/10 mg is one capsule daily by mouth for the initial seven days of therapy. On the eighth day of therapy and thereafter, the daily dose should be a one capsule every 12 hours for a total of two capsules daily. The need for continued treatment should be reassessed periodically, as spontaneous improvement of PBA occurs in some patients.

The most common adverse reactions (incidence of ≥3% and two-fold greater than placebo) in patients taking NUEDEXTA® in descending order are diarrhea, dizziness, cough, vomiting, asthenia, peripheral edema, urinary tract infection, influenza, increased gamma glutamyltransferase, and flatulence. The following adverse reactions have been reported with the use of the individual component dextromethorphan: drowsiness, dizziness, nervousness or restlessness, nausea, vomiting, and stomach pain.

### BRIEF SUMMARY

In various embodiments, the present disclosure is based in part on the unexpected discovery that it is possible to administer dextromethorphan transdermally with a sustained high flux of dextromethorphan, such as for a period of seven days or more, from the transdermal delivery devices (patches) herein, which typically contains dextromethorphan in a solid state dispersed in the adhesive layer and/or the reservoir layer. The transdermal delivery of dextromethorphan herein achieves therapeutically effective plasma concentrations of dextromethorphan, for example, for treating a disease or disorder herein, such as PBA. Compared to the currently available oral delivery through formulations such as Nuedexta®, the transdermal delivery of dextromethorphan herein has numerous advantages and solves many of the unmet medical needs of such oral formulations. For example, the transdermal delivery device or formulations (e.g., adhesive compositions) herein can be administered to achieve a therapeutically effective plasma concentration without regard to whether a CYP2D6 inhibitor such as quinidine is co-administered. As such, the transdermal delivery devices or formulations herein can be administered to transdermally deliver dextromethorphan to subjects who are sensitive or intolerant to CYP2D6 inhibitors such as quinidine. The transdermal delivery devices or formulations herein can be conveniently administered to transdermally deliver dextromethorphan to a subject with or without first determining whether the subject is a poor metabolizer, an intermediate metabolizer, or an extensive metabolizer of dextromethorphan. Administering dextromethorphan using the transdermal delivery devices or formulations herein can also provide superior clinical experience compared to Nuedexta®, for example, with more accurate dosing, less frequent dosing, reduced potential for side effects associated with quinidine and/or higher exposure (e.g., Cₘₐₓ) of dextromethorphan, reduced pill burden, and better patient compliance.

In various embodiments, provided herein are novel transdermal delivery devices (or patches) comprising dextromethorphan, pharmaceutical compositions (e.g., transdermal formulations such as adhesive compositions) comprising dextromethorphan, methods of preparation thereof, and methods of administering dextromethorphan transdermally. The transdermal delivery devices, pharmaceutical compositions, and methods herein are useful in treating various diseases and disorders such as neurological diseases or disorders (e.g., PBA).

In some embodiments, the present disclosure provides:
[1] A transdermal patch comprises:
   i. a backing layer; and
   ii. a drug-in-adhesive layer comprising 1) dextromethorphan in an amount of at least 15%, preferably, about 15% to about 50% by weight; 2) isopropyl myristate in an amount of about 6% to about 12% by weight; 3) a pressure sensitive adhesive, preferably, an acrylate based pressure sensitive adhesive, in an amount of about 20% to about 60% by weight; and 4) a solubilizer in an amount of about 6% to about 25% by weight,
wherein at least a portion of the dextromethorphan is in a solid state dispersed in the pressure sensitive adhesive, preferably, the transdermal patch has an active surface area of about 30 cm² to about 150 cm².
[2] The transdermal patch of [1], wherein the acrylate based pressure sensitive adhesive is an acrylate copolymer adhesive, e.g., a poly acrylate vinyl acetate copolymer pressure sensitive adhesive, such as those having non-acidic hydroxyl functional groups, for example, described herein such as Duro-Tak 87-2287 adhesive and the alike.
[3] The transdermal patch of [1] or [2], wherein the acrylate based pressure sensitive adhesive is in an amount of about 20%, about 30%, about 40%, about 45%, about 50%, about 55%, or about 60%, by weight, or any values or ranges between the recited values, such as about 40-60%, about 45-55% etc.
[4] The transdermal patch of any of [1]-[3], wherein the solubilizer is a vinylpyrrolidone polymer, such as vinyl pyrrolidone homopolymer (or povidone), for example, Povidone K30, Plasdone K29/32 and the alike.
[5] The transdermal patch of any of [1]-[4], wherein the solubilizer is present in an amount of about 6%, about 8%, about 10%, about 15%, about 20%, or about 25%, or any values or ranges between the recited values, such as about 8-15% or about 6-20% etc.
[6] The transdermal patch of any of [1]-[5], wherein the isopropyl myristate is in an amount of about 6%, about 7%, about 8%, about 9%, about 10%, about 11%, or about 12%, by weight, or any values or ranges between the recited values, such as about 8-12% etc.
[7] The transdermal patch of any of [1]-[6], wherein the dextromethorphan is in an amount of about 15%, about 20%, about 25%, about 30%, about 35%, about 40%, about 45%, or about 50%, by weight, or any values or ranges between the recited values, such as about 20-40%, about 25-35% etc.
[8] The transdermal patch of any of [1]-[7], wherein the drug-in-adhesive layer comprises about 150 mg to about 1000 mg of dextromethorphan, e.g., about 200 mg, about 250 mg, about 300 mg, about 350 mg, about 400 mg, about 450 mg, about 500 mg, about 600 mg, about 750 mg, about 900 mg, about 1000 mg, or any values or ranges between the recited values, such as about 250-400 mg, about 300-400 mg, or about 350-450 mg, or about 300-800 mg, about 350-900 mg, etc. of dextromethorphan.
[9] The transdermal patch of any of [1]-[8], wherein the drug-in-adhesive layer comprises about 30 mg to about 350 mg of isopropyl myristate, e.g., about 30 mg, about 50 mg, about 75 mg, about 100 mg, about 125 mg, about 150 mg, about 175 mg, about 200 mg, about 250 mg, about 300 mg, about 350 mg, or any values or ranges between the recited values, such as about 50-150 mg or about 75-125 mg, about 75-300 mg, about 50-250 mg, etc. of isopropyl myristate.
[10] The transdermal patch of any of [1]-[9], wherein the drug-in-adhesive layer comprises about 150 mg to about 1800 mg of the pressure sensitive adhesive, preferably acrylate based pressure sensitive adhesive, e.g., about 300 mg, about 350 mg, about 400 mg, about 450 mg, about 500 mg, about 550 mg, about 600 mg, about 700 mg, about 800 mg, about 900 mg, about 1000 mg, about 1200 mg, about 1400 mg, about 1600 mg, about 1800 mg, or any values or ranges between the recited values, such as about 350-600 mg, about 400-650 mg, or about 450-700 mg, about 450-1200 mg, about 350-1000 mg, about 400-1600 mg, etc. of the pressure sensitive adhesive.
[11] The transdermal patch of any of [1]-[10], wherein the drug-in-adhesive layer comprises the solubilizer in an amount of about 30 mg to about 350 mg, e.g., in an amount of e.g., about 30 mg, about 50 mg, about 75 mg, about 100 mg, about 125 mg, about 150 mg, about 175 mg, about 200 mg, about 250 mg, about 300 mg, about 350 mg, or any values or ranges between the recited values, such as about 50-150 mg or about 75-125 mg, about 75-300 mg, about 50-250 mg, etc.
[12] The transdermal patch of any of [1]-[11], wherein the drug-in-adhesive layer comprises dextromethorphan as the only active ingredient.
[13] The transdermal patch of any of [1]-[12], which has an active surface area of about 70 cm².
[14] The transdermal patch of any of [1]-[13], which has a total dextromethorphan loading of about 2 mg/cm² to about 25 mg/cm², such as about 2.5 mg/cm², about 3 mg/cm², about 4 mg/cm², about 5 mg/cm², about 6 mg/cm², about 7 mg/cm², about 8 mg/cm², about 9 mg/cm², about 10 mg/cm², about 12 mg/cm², about 15 mg/cm², about 20 mg/cm², about 25 mg/cm², or any values or ranges between the recited values, such as about 3-10 mg/cm², about 4-7 mg/cm², about 5-12 mg/cm², about 8-25 mg/cm², etc.
[15] The transdermal patch of any of [1]-[14], which consists of the backing layer, drug-in-adhesive layer, and optionally a release liner.
[16] The transdermal patch of any of [1]-[15], in the form of a single layer patch.
[17] The transdermal patch of any of [1]-[16], wherein the transdermal patch has a dextromethorphan flux of at least about 200 ug/cm²/day for at least seven days, when measured in vitro using human cadaver skin, such as about 200 ug/cm²/day, about 300 ug/cm²/day, about 400 ug/cm²/day, about 500 ug/cm²/day, about 600 ug/cm²/day, about 700 ug/cm²/day, about 800 ug/cm²/day, about 1000 ug/cm²/day, or any values or ranges between the recited values, such as about 200-800 ug/cm²/day, about 300-800 ug/cm²/day, about 400-800 ug/cm²/day, about 500-800 ug/cm²/day, etc., for seven days or more.
[18] The transdermal patch of any of [1]-[16], wherein a single application of the transdermal patch to a human subject is capable of transdermally delivering a daily dose of about 15 mg to about 50 mg (e.g., about 15 mg, about 20 mg, about 30 mg, about 40 mg, about 50 mg, or any values or ranges between the recited values, such as about 20-50 mg, about 30-50 mg, or about 20-40 mg, etc.) of dextromethorphan to the human subject for at least seven days.
[19] The transdermal patch of any of [1]-[16], wherein a single application of the transdermal patch to a human subject is capable of transdermally delivering a therapeutically effective daily dose for treating pseudobulbar affect to the human subject for at least seven days.
[20] A transdermal patch comprising:
   i. a backing layer;
   ii. a skin-contacting drug-in-adhesive layer comprising 1) dextromethorphan in an amount of about 6% to about 12% by weight; 2) isopropyl myristate in an amount of about 6% to about 12% by weight; 3) a first pressure sensitive adhesive, preferably, an acrylate based pressure sensitive adhesive, in an amount of about 65% to about 85% by weight; and 4) a crystallization inhibitor in an amount of about 6% to about 12% by weight, and
   iii. a reservoir layer comprising 1) dextromethorphan in an amount of at least 15% (e.g., about 15% to about 50%) by weight; 2) isopropyl myristate in an amount of about 6% to about 12% by weight; (3) a second pressure sensitive adhesive in an amount of about 20% to about 60% by weight; and (4) a solubilizer in an amount of about 6% to about 25% by weight,
wherein at least a portion of the dextromethorphan in the reservoir layer is in a solid state dispersed in the second pressure sensitive adhesive, and the first and second pressure sensitive adhesive are the same or different, preferably the same, preferably,
the transdermal patch has an active surface area of about 30 cm² to about 150 cm².
[21] The transdermal patch of claim [20], wherein the first pressure sensitive adhesive is an acrylate based pressure sensitive adhesive.
[22] The transdermal patch of [21], wherein the acrylate based pressure sensitive adhesive is an acrylate copolymer adhesive, e.g., a poly acrylate vinyl acetate copolymer pressure sensitive adhesive, such as those having non-acidic hydroxyl functional groups, for example, described herein such as Duro-Tak 87-2287 adhesive and the alike.
[23] The transdermal patch of [21] or [22], wherein the acrylate based pressure sensitive adhesive is in an amount of about 65%, about 70%, about 75%, about 80%, or about 85%, by weight, or any values or ranges between the recited values, such as about 70-85%, about 65-75% etc., of the skin-contacting drug-in-adhesive layer.
[24] The transdermal patch of any of [20]-[23], wherein the crystallization inhibitor is a vinylpyrrolidone polymer, such as vinyl pyrrolidone homopolymer (or povidone), for example, Povidone K30, Plasdone K29/32 and the alike.
[25] The transdermal patch of any of [20]-[24], wherein the crystallization inhibitor is present in an amount of about 6%, about 7%, about 8%, about 9%, about 10%, about 11%, or about 12%, or any values or ranges between the recited values, such as about 6-12% or 8-12% etc., of the skin-contacting drug-in-adhesive layer.
[26] The transdermal patch of any of [20]-[25], wherein the isopropyl myristate is in an amount of about 6%, about 7%, about 8%, about 9%, about 10%, about 11%, or about 12%, by weight, or any values or ranges between the recited values, such as about 8-12% etc., of the skin-contacting drug-in-adhesive layer.
[27] The transdermal patch of any of [20]-[26], wherein the dextromethorphan in the skin-contacting drug-in-adhesive layer is in an amount of about 6%, about 7%, about 8%, about 9%, about 10%, about 11%, or about 12%, by weight, or any values or ranges between the recited values, such as about 6-10% etc., of the skin-contacting drug-in-adhesive layer.
[28] The transdermal patch of any of [20]-[27], wherein the second pressure sensitive adhesive is an acrylate based pressure sensitive adhesive.
[29] The transdermal patch of any of [20]-[28], wherein the second pressure sensitive adhesive is an acrylate copolymer adhesive, e.g., a poly acrylate vinyl acetate copolymer pressure sensitive adhesive, such as those having non-acidic hydroxyl functional groups, for example, described herein such as Duro-Tak 87-2287 adhesive and the alike.
[30] The transdermal patch of any of [20]-[29], wherein the second pressure sensitive adhesive is in an amount of about 20%, about 30%, about 35%, about 40%, about 45%, about 50%, or about 60%, by weight, or any values or ranges between the recited values, such as about 35-45%, about 30-50% etc., of the reservoir layer.
[31] The transdermal patch of any of [20]-[30], wherein the solubilizer is a vinylpyrrolidone polymer, such as vinyl pyrrolidone homopolymer (or povidone), for example, Povidone K30, Plasdone K29/32 and the alike.
[32] The transdermal patch of any of [20]-[31], wherein the solubilizer is present in an amount of about 6%, about 8%, about 10%, about 15%, about 20%, or about 25%, or any values or ranges between the recited values, such as about 15-25% or about 10-20% etc., of the reservoir layer.
[33] The transdermal patch of any of [20]-[32], wherein the isopropyl myristate in the reservoir layer is in an amount of about 6%, about 7%, about 8%, about 9%, about 10%, about 11%, or about 12%, by weight, or any values or ranges between the recited values, such as about 8-12% etc., of the reservoir layer.
[34] The transdermal patch of any of [20]-[33], wherein the dextromethorphan in the reservoir layer is in an amount of about 15%, about 20%, about 25%, about 30%, about 35%, about 40%, about 45%, or about 50%, by weight, or any values or ranges between the recited values, such as about 20-40%, about 25-35% etc., of the reservoir layer.
[35] The transdermal patch of any of [20]-[34], wherein the dextromethorphan in the reservoir layer has a concentration that is about 200-800% (preferably, about 300-500%) of that in the skin contacting drug-in-adhesive layer.
[36] The transdermal patch of any of [20]-[35], wherein the amount of dextromethorphan in the reservoir layer is at least about 200%, preferably, about 200-3000% (preferably, about 300-1500%) of that in the skin contacting drug-in-adhesive layer.
[37] The transdermal patch of any of [20]-[36], wherein the skin contacting drug-in-adhesive layer has a thickness of about 0.4 mil to about 5 mils, such as about 0.4-3 mils.
[38] The transdermal patch of any of [20]-[37], wherein the reservoir layer has a thickness of about 2 mils to about 7.5 mils such as about 4 mils.
[39] The transdermal patch of any of [20]-[38], wherein the reservoir layer has a thickness of at least about 100%, preferably, about 100-800% (preferably, 100-600% or 120-300%) of that of the skin contacting drug-in-adhesive layer.
[40] The transdermal patch of any of [20]-[39], wherein the total weight of the reservoir layer is at least about 100%, about 100-800% (preferably, about 100-600% or about 120-300%) of that of the skin contacting drug-in-adhesive layer.
[41] The transdermal patch of any of [20]-[40], wherein the total amount of dextromethorphan is about 150 mg to about 1000 mg, e.g., about 200 mg, about 250 mg, about 300 mg, about 350 mg, about 400 mg, about 450 mg, about 500 mg, about 600 mg, about 750 mg, about 900 mg, about 1000 mg, or any values or ranges between the recited values, such as about 250-400 mg, about 300-400 mg, or about 350-450 mg, or about 300-800 mg, about 350-900 mg, etc.
[42] The transdermal patch of any of [20]-[41], wherein the total amount of isopropyl myristate is about 30 mg to about 350 mg, e.g., about 30 mg, about 50 mg, about 75 mg, about 100 mg, about 125 mg, about 150 mg, about 175 mg, about 200 mg, about 250 mg, about 300 mg, about 350 mg, or any values or ranges between the recited values, such as about 50-150 mg or about 75-125 mg, about 75-300 mg, about 50-250 mg, etc.
[43] The transdermal patch of any of [20]-[42], wherein the combined amount of the first and second pressure sensitive adhesive is about 150 mg to about 1800 mg, e.g., about 300 mg, about 350 mg, about 400 mg, about 450 mg, about 500 mg, about 550 mg, about 600 mg, about 700 mg, about 800 mg, about 900 mg, about 1000 mg, about 1200 mg, about 1400 mg, about 1600 mg, about 1800 mg, or any values or ranges between the recited values, such as about 350-600 mg, about 400-650 mg, or about 450-700 mg, about 450-1200 mg, about 350-1000 mg, about 400-1600 mg, etc.
[44] The transdermal patch of any of [20]-[43], comprising dextromethorphan as the only active ingredient.
[45] The transdermal patch of any of [20]-[44], which has an active surface area of about 70 cm².
[46] The transdermal patch of any of [20]-[45], which has a total dextromethorphan loading of about 2 mg/cm² to about 25 mg/cm², such as about 2.5 mg/cm², about 3 mg/cm², about 4 mg/cm², about 5 mg/cm², about 6 mg/cm², about 7 mg/cm², about 8 mg/cm², about 9 mg/cm², about 10 mg/cm², about 12 mg/cm², about 15 mg/cm², about 20 mg/cm², about 25 mg/cm², or any values or ranges between the recited values, such as about 3-10 mg/cm², about 4-7 mg/cm², about 5-12 mg/cm², about 8-25 mg/cm², etc.
[47] The transdermal patch of any of [20]-[46], which consists of the backing layer, skin contacting drug-in-adhesive layer, reservoir layer, and optionally a release liner.
[48] The transdermal patch of any of [20]-[47], in the form of a bilayer patch.
[49] The transdermal patch of any of [20]-[48], wherein the transdermal patch has a dextromethorphan flux of at least about 200 ug/cm²/day for at least seven days, when measured in vitro using human cadaver skin, such as about 200 ug/cm²/day, about 300 ug/cm²/day, about 400 ug/cm²/day, about 500 ug/cm²/day, about 600 ug/cm²/day, about 700 ug/cm²/day, about 800 ug/cm²/day, about 1000 ug/cm²/day, or any values or ranges between the recited values, such as about 200-800 ug/cm²/day, about 300-800 ug/cm²/day, about 400-800 ug/cm²/day, about 500-800 ug/cm²/day, etc., for seven days or more.
[50] The transdermal patch of any of [20]-[48], wherein a single application of the transdermal patch to a human subject is capable of transdermally delivering a daily dose of about 15 mg to about 50 mg (e.g., about 15 mg, about 20 mg, about 30 mg, about 40 mg, about 50 mg, or any values or ranges between the recited values, such as about 20-50 mg, about 30-50 mg, or about 20-40 mg, etc.) of dextromethorphan to the human subject for at least seven days.
[51] The transdermal patch of any of [20]-[48], wherein a single application of the transdermal patch to a human subject is capable of transdermally delivering a therapeutically effective daily dose for treating pseudobulbar affect to the human subject for at least seven days.
[52] A method of treating a neurological disease or disorder (e.g., any of those described herein) in a subject in need thereof, the method comprising applying the transdermal patch of any one of [1]-[51] to the subject.
[53] The method of [52], comprising applying the transdermal patch to transdermally deliver a therapeutically effective amount of dextromethorphan to the subject.
[54] The method of [52], comprising applying the transdermal patch to transdermally deliver a daily dose of about 15 mg to about 50 mg (e.g., about 15 mg, about 20 mg, about 30 mg, about 40 mg, about 50 mg, or any values or ranges between the recited values, such as about 20-50 mg, about 30-50 mg, or about 20-40 mg, etc.) of dextromethorphan to the subject.
[55] The method of [54], wherein the daily dose is about 35 mg of dextromethorphan.
[56] The method of any one of [52]-[55], wherein the transdermal patch is applied to the subject at a dosing frequency ranging from once a day to once a week, preferably, the transdermal patch is applied to the subject once a week or twice a week.
[57] The method of any one of [52]-[56], wherein the neurological disease or disorder is pseudobulbar affect, depression, such as major depressive disorder or treatment resistant depression, stroke, traumatic brain injury, seizure, pain, methotrexate neurotoxicity, Parkinson's disease, autism, or a combination thereof.
[58] The method of [57], wherein the neurological disease or disorder is pseudobulbar affect.
[59] The method of any one of [52]-[58], wherein the subject does not suffer from a cough and/or does not need an antitussive.
[60] The method of any one of [52]-[59], wherein the subject is characterized as an extensive metabolizer of dextromethorphan.
[61] The method of any one of [52]-[59] wherein the subject is characterized as a poor metabolizer of dextromethorphan.
[62] The method of any one of [52]-[61], wherein the subject is sensitive or intolerant to CYP2D6 inhibitors.
[63] The method of any one of [52]-[62], wherein the subject has one or more side effects associated with quinidine.
[64] The method of any one of [52]-[63], wherein the subject is co-administered a drug whose metabolism is affected by a CYP2D6 inhibitor.
[65] The method of any one of [52]-[64], further comprising administering to the subject an antidepressant.
[66] The method of [65], wherein the antidepressant is selected from bupropion, hydroxybupropion, erythrohydroxybupropion, threohydroxybupropion, a metabolite or prodrug of any of these compounds, and combinations thereof.
[67] The method of any one of [52]-[66], wherein the subject is not administered quinidine.
[68] The method of any one of [52]-[67], wherein the subject is a human subject.

It is to be understood that both the foregoing summary and the following detailed description are exemplary and explanatory only, and are not restrictive of the invention herein.

### BRIEF DESCRIPTION OF THE DRAWINGS/FIGURES

FIG. 1 presents graphs showing in vitro flux study results for transdermal delivery device prepared according to Examples 1 and 2.

### DETAILED DESCRIPTION

Dextromethorphan (DXM) has been used orally to treat neurological disorders such as pseudobulbar affect (PBA), emotional lability, agitation in Alzheimer's, major depressive disorder, treatment resistant disorder, pain management, other CNS disorders, and the like. But, to be effective, it must be delivered with a substance that competitively inhibits the liver enzyme cytochrome P450 2D6 (CYP2D6). It particular, this has meant it is co-administered with quinidine. Otherwise, too little makes it pass the liver's diligence of digested food.

The present disclosure generally relates to transdermal delivery of dextromethorphan using the transdermal delivery devices, formulations (e.g., adhesive compositions), and methods herein, which provides many advantages over the currently available oral formulations (e.g., Nuedexta®) and solves many unmet medical needs of such oral formulations. For example, the transdermal delivery devices or formulations herein can be administered to achieve a therapeutically effective plasma concentration without regard to whether a CYP2D6 inhibitor such as quinidine is co-administered. As such, the transdermal delivery devices or formulations herein can be administered to transdermally deliver dextromethorphan to subjects who are for example, sensitive or intolerant to CYP2D6 inhibitors such as quinidine (e.g., having one or more side effects associated with quinidine, or is co-administered a drug whose metabolism is affected by CYP2D6 inhibitors such as quinidine). Further, the transdermal delivery devices or formulations herein can be conveniently administered to transdermally deliver dextromethorphan to a subject with or without first determining whether the subject is a poor metabolizer, an intermediate metabolizer, or an extensive metabolizer of dextromethorphan. For brevity, as used herein, unless otherwise obvious from context, poor metabolizer (PM), intermediate metabolizer (IM), or extensive metabolizer (EM) refers to the subject's ability to metabolize dextromethorphan. Categorization of a subject as a PM, IM, or EM (alternatively labeled as ultrametabolizers or ultrarapid metabolizers or UM) is known in the art. See e.g., Treducu A.L.D. et al. Frontiers in Pharmacology, vol. 9, Article 305 (April 2018), which based on genotype assigned subjects as UM if containing “≥3 normal function gene copies”).

Administering dextromethorphan using the transdermal delivery devices or formulations herein can also provide superior clinical experience compared to Nuedexta®, for example, with more accurate dosing, less frequent dosing, reduced potential for side effects associated with quinidine and/or higher exposure (e.g., Cₘₐₓ) of dextromethorphan, reduced pill burden, and better patient compliance. In view of this disclosure, those skilled in the art could select a proper patch to more precisely deliver a therapeutically effective amount of dextromethorphan to the subject treated. Additionally, the PK profile described herein and in WO 2021/202329 A1 shows that transdermal delivery of dextromethorphan can achieve a much lower but effective amount of dextromethorphan plasma exposure compared to a twice-a-day oral dosing of Nuedexta® tablets. Thus, it is expected that the methods herein would at least produce a reduced incidence of side effects associated with high exposure (e.g., Cₘₐₓ, AUC, etc.) of dextromethorphan. The transdermal delivery devices herein can be configured as a 1-day patch, 2-day patch, 3-day patch, 4-day patch, 5-day patch, 6-day patch, or 7-day patch, which is suitable for dosing frequencies ranging from once a day to once a week, for example, once in more than 24 hours, more than 36 hours, more than 48 hours, etc., or 1, 2, 3, 4, 5, or 6 times a week. Using the transdermal delivery devices herein can provide improved patient compliance, at least by avoiding the twice-a-day dosing regimen of Nuedexta®.

Prior to Applicant's work, it was not known whether dextromethorphan can be delivered transdermally to achieve a therapeutically effective plasma concentration for treating a neurological disease or disorder such as PBA. The unpredictability of transdermal administration is notorious. For example, testosterone can be delivered transdermally without enhancer at a rate three orders of magnitude higher than for beta estradiol. Structurally and by calculated LogP, these compounds are very similar, such that this difference could not be anticipated. See, U.S. Provisional Appl. No. 62/568,028, filed Oct. 4, 2017, the content of which is incorporated by reference in its entirety. U.S. Pat. No. 6,335,030 B1 describes some examples of dextromethorphan patches with a goal to achieve an antitussive effect. However, no pharmacokinetic data on transdermal administration of dextromethorphan was known before Applicant's work described in U.S. Provisional Application 62/680,182 and International Application No. PCT/US2018/054178, published as WO2019/070864, the content of each of which is herein incorporated by reference in its entirety.

In PCT/US2018/054178, it was shown that transdermal delivery of dextromethorphan, without using quinidine, can provide a significant blood level of dextromethorphan in human. PCT/US2018/054178 describes a human pharmacokinetic study showing that applying to healthy human an exemplary patch containing about 35 mg dextromethorphan with a size of 45 cm², which was designed to transdermally deliver 15 mg per day and contains, in the adhesive layer (drug-in-adhesive layer) about 80% by weight of an adhesive (Duro-Tak 87-2287), about 10% by weight of dextromethorphan base and about 10% by weight of permeation enhancer isopropyl myristate, for about 24 hours, achieved, inter alia, a mean Cₘₐₓ of about 6 ng/ml and a mean AUC₀₋₂₄ h of about 92 h·ng/mL, approaching those observed from orally administering Nuedexta® tablets (a combination of 20 mg dextromethorphan and 10 mg quinidine) twice a day to the human subject.

Further developments in WO 2021/202329, the content of which is herein incorporated by reference in its entirety, show that the inclusion of a crystallization inhibitor, a vinylpyrrolidone polymer (Plasdone K29/32) in dextromethorphan transdermal patches significantly enhanced the permeation of dextromethorphan from the patches, in vitro and in vivo. In particular, it was shown that a 70 cm² patch applied for 24 hours can deliver a daily dose of about 32.4 mg to about 41.1 mg of dextromethorphan to human subjects, which therefore has a flux of dextromethorphan of about 0.46 mg/cm²/day to about 0.59 mg/cm²/day. This represents a significantly higher flux compared to a similar patch, except without the vinylpyrrolidone polymer (replaced with the adhesive matrix Duro-Tak 87-2287), which has an estimated flux of about 0.33 mg/cm²/day. Additional in vivo data also indicates that the per unit patch area (cm²) delivery of dextromethorphan is enhanced with patches having the vinylpyrrolidone polymer.

The examples shown in WO2019/070864 and WO 2021/202329 concern mainly transdermal patches that do not contain drug particles or dextromethorphan in a solid state, such as dextromethorphan crystals. It was believed that these drug crystals would impede dextromethorphan permeation and are not desired. However, as shown herein, it was unexpectedly found that with certain combinations of ingredients, dextromethorphan can be included in the transdermal patch at high concentrations and can exist at least partially in a solid state (such as dextromethorphan crystals), and the transdermal patch can have a desired permeation of dextromethorphan for an extended period of time such that the transdermal patch is suitable for a once a week dosing regimen.

In various embodiments, the present disclosure provides transdermal delivery devices and formulations comprising a high concentration of dextromethorphan, methods of preparing the same, methods of delivering dextromethorphan transdermally using the transdermal delivery devices or formulations herein, and methods of treating a disease or disorder using the transdermal delivery devices or formulations herein.

### Transdermal Delivery Device Comprising Dextromethorphan

Certain embodiments of the present disclosure are directed to novel transdermal delivery devices comprising dextromethorphan.

Various patch designs can be used for the transdermal delivery device herein. The transdermal delivery device herein typically comprises a backing layer, an adhesive layer (e.g., a drug-in-adhesive layer), which is the skin-contacting layer when in use, and optionally a reservoir layer. The adhesive layer typically comprises dextromethorphan dispersed in an adhesive, preferably a pressure sensitive adhesive. More than one adhesive layers can be used for the transdermal delivery device herein. The adhesive layer is typically formulated such that the transdermal delivery device can adhere to the skin of a user for a desired period of time. For example, in some embodiments, the transdermal delivery device is capable of adhering continuously to the skin of a user for about 8 hours, about 12 hours, about 18 hours, about 24 hours, about 2 days, about 3 days, about 4 days, about 5 days, about 6 days, or about 7 days or more.

In some embodiments, the transdermal delivery device can be a drug-in-adhesive (DIA) patch. In some embodiments, the DIA patch is a single layer patch, for example, the single layer includes dextromethorphan dispersed in the adhesive, in which at least a portion of dextromethorphan exists in a solid state, i.e., not fully dissolved in the adhesive, or maybe alternatively referred to as having dextromethorphan particles, which can be dextromethorphan crystals.

In some embodiments, the DIA patch is a multilayer patch. For example, two drug-in-adhesive layers can be included in the patch, which is optionally separated by a membrane, e.g., a rate-controlling membrane, or by a reservoir layer. In some embodiments, the two drug-in-adhesive layers can sandwich a reservoir layer. In some embodiments, one of the drug-in-adhesive layers is the skin-contacting drug-in-adhesive layer and the other of the drug-in-adhesive layers can be a reservoir layer, for example, with a higher dextromethorphan concentration than the skin-contacting drug-in-adhesive layer. In such embodiments, the reservoir layer typically includes at least a portion of the dextromethorphan in a solid state dispersed in the adhesive.

The transdermal delivery device can include dextromethorphan as the only drug or in combination with another drug. Unless obviously contradictory, in any of the embodiments described herein, dextromethorphan can be the only drug in the transdermal delivery device. Dextromethorphan can exist in various forms, for example, as a free base or a pharmaceutically acceptable salt. As used herein, the weight percentage, concentration, flux, etc. regarding dextromethorphan should be understood as the total amount of dextromethorphan measured and/or calculated, with the value expressed in the equivalent value for dextromethorphan base. Further, all weight percentages or thickness, unless otherwise obvious from context, should refer to the weight percentage or thickness based on the final formulation (e.g., final adhesive layer or reservoir layer etc.) or transdermal delivery device as appropriate. In any of the embodiments described herein, the dextromethorphan can exist in its free base form, except that it can be protonated through equilibrium with other ingredient(s). For example, in any of the embodiments described herein, the transdermal delivery device or pharmaceutical compositions described herein can be prepared by mixing directly or indirectly the recited amount of dextromethorphan base with the other ingredients.

In any of the embodiments described herein, the dextromethorphan in the transdermal delivery device can be partially or completely replaced with a deuterated dextromethorphan, e.g., the d3 analog (O—CD₃, or N—CD₃) or d6 analog (N-CD₃,O—CD₃) see, e.g., claims 1 and 17 of U.S. Pat. No. 7,973,049, the content of which is incorporated by reference in its entirety. Apparently, in such embodiments, the methods using the deuterated dextromethorphan patches would provide deuterated dextromethorphan to the user. As used herein, a deuterated dextromethorphan refers to a compound resulted from substituting one or more hydrogen atoms of dextromethorphan with deuterium such that each substituted position has a deuterium content above the natural abundance, i.e., the substituted position is enriched with deuterium. In some embodiments, the deuterated dextromethorphan has at least one position with deuterium enriched to at least 10% deuterium, at least 50% deuterium, at least 90% deuterium, at least 95% deuterium or at least 98% deuterium. In any of the embodiments described herein, the dextromethorphan in the transdermal delivery device can also be partially or completely replaced with a dextromethorphan analog, such as a fluorinated dextromethorphan or a skin permeable prodrug of dextromethorphan, etc.

The adhesive layer typically includes a pressure sensitive adhesive (PSA). Useful features for pressure sensitive adhesive include adequate tack, good adhesion and cohesive strength. Further useful attributes include biocompatibility (e.g., non-irritating, non-sensitizing non-toxic), formulation compatibility, delivery system compatibility and the like. Useful pressure sensitive adhesive include for example polyacrylates, poly acrylic esters, silicones, polyisobutylenes and the like.

PSAs are generally known in the art. See, e.g., Tan et al., Pharm Sci & Tech Today, 2:60-69 (1999). Non-limiting useful PSAs include polyisobutylenes (PIB), silicone polymers, acrylate copolymers, and combinations thereof. In some embodiments, the pressure sensitive adhesive comprises a polyisobutylene adhesive, a silicone polymer adhesive, an acrylate copolymer adhesive, or a combination thereof. In some embodiments, the pressure sensitive adhesive comprises an acrylate copolymer adhesive. Non-limiting useful acrylate copolymers include, for example, acrylic pressure sensitive adhesives such as a poly acrylate vinyl acetate copolymer, e.g., Duro-Tak 87-2287, Duro-Tak 87-4098, Duro-Tak 87-4287, or Duro-Tak 87-2516, Duro-Tak 87-2852 or Duro-Tak 87-2194, which are manufactured by Henkel Adhesives. PIBs are elastomeric polymers that are commonly used in PSAs, both as primary-base polymers and as tackifiers. PIBs are homopolymers of isobutylene and feature a regular structure of a carbon-hydrogen backbone with only terminal unsaturation. Non-limiting useful PIBs include those marketed under the trade name Oppanol by BASF. The silicone polymers are a high molecular weight polydimethylsiloxane that contains residual silanol functionality (SiOH) on the ends of the polymer chains. Non-limiting useful silicone PSAs for use in pharmaceutical applications include those available from Dow Corning Corporation, for example under the trade name of BIO-PSA, e.g., BIO-7-4202. In some embodiments, the adhesive layer is about 0.1 mils to about 10 mils, e.g., about 1.5 mils to about 10 mils (e.g., about 1.5 mils to about 2 mils) thick.

In some embodiments, suitable adhesives include for example the following silicone adhesives from Dow Corning: BIO-PSA 7-410X, BIO-PSA 7-420X, BIO-PSA 7-430X, BIO-PSA 7-440X, BIO-PSA 7-450X, BIO-PSA 7-460X, and BIO-PSA Hot Melt Adhesive. In some embodiments, suitable adhesives include for example the following polyacrylate/poly acrylic ester adhesives from Henkel Adhesives: Duro-Tak 87-900A, 87-9301, 87-4098, 87-2510, 87-2287, 87-2677, 87-4287, 87-2516, 87-2074, 87-235A, 87-2353, 87-2852, 87-2051, 87-2052, 87-2054, 87-2194, 87-2196, 87-6908, 387-2510, 387-2287, 387-2516, 387-2353, 387-2051, 387-2051 and 387-2054, GELVA GMS 3083, 3253, 788 and 9073. These can for example have hydroxyl functional groups, carboxylic groups, hydroxyl and carboxylic groups, or no functional groups (as active as the foregoing). These can for example include vinyl acetate monomer, or not. In some embodiments, the pressure sensitive adhesive can be copolymers formed from acrylate monomers and vinyl acetate, including those containing non-acidic hydroxyl functional groups, such as DuroTak® 2287 (87-2287, 387-2287, etc.) adhesives and the alike. DuroTak® 87-2287 and 387-2287 have the same polymer composition, which is used herein interchangeably. A typical composition of DuroTak® 2287 can include random copolymers formed from the following monomers: 2-ethylhexylacrylate (e.g., about 68.2%), vinyl acetate (e.g., about 26.5%), hydroxyethylacrylate (e.g., about 5.2%), and glycidylmethacrylate (e.g., about 0.15%). In some embodiments, the acrylate copolymer adhesive can be formed from monomers including about 5.2 wt % 2-hydroxyethyl acrylate, about 20-40 wt % vinyl acetate, and about 55-75 wt % 2-ethylhexyl acrylate. See also U.S. Published Application No. US20060257462A1 and U.S. Pat. No. 5,693,335, the content of each of which is herein incorporated by reference in its entirety.

Typically, the transdermal delivery device herein is supported by a backing layer such as an impermeable backing film, and the adhesive surface is protected by a release liner prior to use. Various materials can be used as a backing layer for the transdermal delivery device herein. Typically, the backing layer is impermeable. For example, the backing layer can be comprised of impermeable polymeric films such as polyester (PET) or polyethylene (PE) films. In some embodiments, the backing layer can comprise a polyester, such as Scotchpak 9736 or Scotchpak 1012, a polyurethane film, such as Scotchpak 9701, or a polyethylene film, such as CoTran 9720. In some embodiments, the backing is part of an overlay, and can be a non-woven fabric, a polyurethane film, or other pliable material to provide flexibility and better wear.

The release liner can be manufactured in the desired size for the present invention. The release liner can be comprised of silicone or fluoro-polymer coated polyester film. The release liner protects the transdermal delivery device during storage and is removed before its use. Silicone-coated release liners include those manufactured by Mylan Corporation, Loparex Corporation, and 3M's Drug Delivery Systems. The fluoro-polymer coated release liners include those manufactured and supplied by 3M's Drug Delivery Systems and Loparex. In some embodiments, the release liner comprises 3M's ScotchPak 9744 or Scotchpak 1022.

The transdermal delivery devices herein can also optionally include other suitable excipients such as humectants, plasticizers, antioxidants, anti-irritants, gel-forming agents, crystallization inhibitors, drug release modifiers etc. These excipients are within the knowledge of those skilled in the art, and can be found, for example, in the Handbook of Pharmaceutical Excipients, (7th ed. 2012), the entire content of which is hereby incorporated by reference. In some embodiments, additional active ingredient(s) can also be included in the transdermal delivery device herein.

The transdermal delivery devices herein can have different sizes (patch sizes) depending on its application. Typically, the patch sizes can be about 5 cm² to about 300 cm² (e.g., about 5 cm², about 10 cm², about 20 cm², about 30 cm², about 40 cm², about 50 cm², about 60 cm², about 80 cm², about 100 cm², about 120 cm², about 150 cm², about 200 cm² or any values or ranges between the specified values), for example, about 30 cm² to about 150 cm², about 10 cm² to about 100 cm², such as about 70 cm².

When applying the transdermal delivery devices herein to a skin of a subject, all of the adhesive surface can become in contact with the skin in theory. Thus, the area of the adhesive surface defines a skin contact area where the active ingredient from the device can permeate the skin, which is also herein referred to as an active surface area. In some embodiments, the adhesive surface is the only surface of the transdermal delivery device that is in contact with the skin upon application, and the active surface area is the same as the area of the adhesive surface. In some embodiments, the adhesive surface and one or more other surfaces of the transdermal delivery device are in contact with the skin upon application, and the entire skin contact area is the active surface area. In a typical DIA patch, the patch size is the same as the active surface area. However, as would be understood by those of ordinary skill in the art, when an overlay design (an overlay typically contains adhesive and/or other ingredient but is essentially free or free of active ingredient) is used, for example, to secure the attachment of the transdermal delivery device to the skin, the adhesive surface from the overlay, although in contact with the skin, does not deliver the active ingredient from the transdermal delivery device. Thus, such adhesive surface from the overlay design is not considered as active surface area. Unless otherwise obvious from context, the unit “/cm²” should be understood as per square centimeter of active surface area as defined herein.

The active surface area can determine the doses of the drug to be delivered.

Typically, the active surface area can be about 5 cm² to about 300 cm² (e.g., about 5 cm², about 10 cm², about 20 cm², about 30 cm², about 40 cm², about 50 cm², about 60 cm², about 80 cm², about 100 cm², about 120 cm², about 150 cm², about 200 cm² or any values or ranges between the specified values), for example, about 30 cm² to about 150 cm², about 10 cm² to about 100 cm², such as about 70 cm².

In some embodiments, the transdermal delivery device herein can be configured to provide dextromethorphan to a user (e.g., human subject) at least about 2 mg/day (e.g., about 2 mg/day to about 50 mg/day) for a period of time of 1 day or more, for example, 2 days, 3 days, 4 days, 5 days, 6 days, or 7 days. For example, in some embodiments, the transdermal delivery device is configured to transdermally deliver dextromethorphan to a user about 5 mg/day to about 50 mg/day (e.g., about 5 mg/day, about 10 mg/day, about 20 mg/day, about 30 mg/day, about 40 mg/day, about 50 mg/day, or any values or ranges between the recited values) for 1 day or more (e.g., 1.5 days, 2 days, 3 days, 4 days, 5 days, 6 days, 7 days, or any values or ranges between the recited values).

In some preferred embodiments, the transdermal delivery device herein (e.g., any of those shown in [1]-[51] in the Brief Summary Section) can be configured such that a single application of the transdermal patch to a human subject is capable of transdermally delivering a daily dose of about 15 mg to about 50 mg (e.g., about 15 mg, about 20 mg, about 30 mg, about 40 mg, about 50 mg, or any values or ranges between the recited values, such as about 20-50 mg, about 30-50 mg, or about 20-40 mg, etc.) of dextromethorphan to the human subject for 7 days or more. In such embodiments, typically, the transdermal patch is applied to the skin of the human subject, and remains adhered to the skin of the human subject for a period of seven days or more, i.e., the patch-on time is about seven days or more until it is taken off or replaced with another transdermal patch. And during the patch-on period, the transdermal patch can transdermally deliver dextromethorphan to the human subject at a substantially same daily dose, for example, the daily dose delivered does not vary from the average daily dose by more than 15 mg/day, more preferably, does not vary by more than 10 mg/day.

In some preferred embodiments, the transdermal delivery device herein (e.g., any of those shown in [1]-[51] in the Brief Summary Section) can be configured such that a single application of the transdermal patch to a human subject is capable of achieving a dextromethorphan flux of at least about 200 ug/cm²/day (“ug” means microgram herein) of dextromethorphan to the human subject for 7 days or more. For example, in some embodiments, a single application of the transdermal patch to a human subject is capable of achieving a dextromethorphan flux of about 200 ug/cm²/day, about 300 ug/cm²/day, about 400 ug/cm²/day, about 500 ug/cm²/day, about 600 ug/cm²/day, about 700 ug/cm²/day, about 800 ug/cm²/day, about 1000 ug/cm²/day, or any values or ranges between the recited values, such as about 200-800 ug/cm²/day, about 300-800 ug/cm²/day, about 400-800 ug/cm²/day, about 500-800 ug/cm²/day, etc., for seven days or more. In some embodiments, during the patch-on period, the transdermal patch can transdermally deliver dextromethorphan to the human subject at a substantially same rate, for example, the cumulative daily permeation of dextromethorphan does not vary from the average cumulative daily permeation of dextromethorphan by more than 350 ug/cm²/day, preferably, does not vary by more than 250 ug/cm²/day, more preferably, does not vary by more than 200 ug/cm²/day.

The total dextromethorphan loading for the transdermal delivery device can be adjusted based on the desired total dose. Typically, the total dextromethorphan loading exceeds 2 mg/cm² (e.g., at least 2 mg/cm², at least 3 mg/cm², at least 4 mg/cm², at least 5 mg/cm², at least 6 mg/cm², at least 7 mg/cm², at least 8 mg/cm², at least 9 mg/cm², at least 10 mg/cm², at least 15 mg/cm², at least 20 mg/cm², at least 25 mg/cm², etc.). For example, in some embodiments, the transdermal delivery device can have a total dextromethorphan loading of about 2 mg/cm² to about 25 mg/cm², such as about 2.5 mg/cm², about 3 mg/cm², about 4 mg/cm², about 5 mg/cm², about 6 mg/cm², about 7 mg/cm², about 8 mg/cm², about 9 mg/cm², about 10 mg/cm², about 12 mg/cm², about 15 mg/cm², about 20 mg/cm², about 25 mg/cm², or any values or ranges between the recited values, such as about 3-10 mg/cm², about 4-7 mg/cm², about 5-12 mg/cm², about 8-25 mg/cm², etc. In some embodiments, the transdermal delivery device can have a total dextromethorphan loading of more than 25 mg/cm², such as up to 30 mg/cm², or up to 35 mg/cm², or beyond. As used herein, the total dextromethorphan loading of a patch can be calculated by dividing the total amount of the dextromethorphan in the patch by the patch's active surface area.

### Exemplary Transdermal Delivery Devices and Formulations

In some embodiments, the present disclosure also provides the following non-limiting exemplary transdermal delivery devices, or alternatively referred to herein as transdermal patches or simply patches, and transdermal formulations such as adhesive compositions.

In some embodiments, the present disclosure provides an adhesive composition comprising (1) dextromethorphan; (2) a pressure sensitive adhesive; (3) a skin permeation enhancer (e.g., isopropyl myristate); and (4) an optional solubilizer, wherein at least a portion of the dextromethorphan is in a solid state (e.g., as dextromethorphan crystals) dispersed in the pressure sensitive adhesive. Although optional, the solubilizer is typically included in the adhesive compositions or drug-in-adhesive layers herein.

In some embodiments, the present disclosure provides an adhesive composition comprising (1) dextromethorphan; (2) a pressure sensitive adhesive; (3) a skin permeation enhancer (e.g., isopropyl myristate); and (4) a solubilizer (e.g., a vinylpyrrolidone polymer, such as vinyl pyrrolidone homopolymer (or povidone), for example, Povidone K30, Plasdone K29/32 and the alike), wherein at least a portion of the dextromethorphan is in a solid state (e.g., as dextromethorphan crystals) dispersed in the pressure sensitive adhesive. A solubilizer as used herein refers to any agent that can facilitate and/or enhance the solubilization of dextromethorphan in the adhesive composition described herein. The soublilizer is typically miscible with the pressure sensitive adhesive in the adhesive composition. In some embodiments, the dextromethorphan is in an amount of at least 15%, preferably, about 15% to about 50% (e.g., about 15%, about 20%, about 25%, about 30%, about 35%, about 40%, about 45%, or about 50%, or any values or ranges between the recited values, such as about 20-40%, about 25-35% etc.) by weight of the adhesive composition; the skin permeation enhancer (e.g., isopropyl myristate) is in an amount of about 6% to about 12% (e.g., about 6%, about 7%, about 8%, about 9%, about 10%, about 11%, about 12%, or any values or ranges between the recited values, such as about 8-12% etc.) by weight of the adhesive composition; the pressure sensitive adhesive is in an amount of about 20% to about 60% (e.g., about 30%, about 40%, about 45%, about 50%, about 55%, or about 60%, by weight, or any values or ranges between the recited values, such as about 40-60%, about 45-55% etc.) by weight of the adhesive composition, and the solubilizer is in an amount of about 6% to about 25% (e.g., about 6%, about 8%, about 10%, about 15%, about 20%, or about 25%, or any values or ranges between the recited values, such as about 8-15% or about 6-20% etc.) by weight of the adhesive composition. While some of the dextromethorphan exists in a solid state and not dissolved in the adhesive composition, the composition of the adhesive composition is typically uniform, for example, the dextromethorphan content across the adhesive composition is typically the same. In some embodiments, the adhesive composition comprises dextromethorphan as the only active ingredient. In some embodiments, the skin permeation enhancer is isopropyl myristate. The pressure sensitive adhesive can be any of those described herein. Typically, the pressure sensitive adhesive is an acrylate copolymer adhesive, e.g., a poly acrylate vinyl acetate copolymer, such as those having non-acidic hydroxyl functional groups, for example, described herein such as Duro-Tak 87-2287 adhesive and the alike, manufactured by Henkel Adhesives. In some embodiments, the pressure sensitive adhesive comprises an acrylate copolymer adhesive and an additional adhesive. For example, in some embodiments, the pressure sensitive adhesive can be a mixture of an acrylate copolymer adhesive (e.g., Durotak 87-2287) and a silicone adhesive (e.g., BIO-7-4202) in various ratios (e.g., a weight ratio of acrylate adhesive to silicone adhesive ranging from about 1:20 to about 20:1, e.g., about 10:1 to about 1:10, such as about 10:1, about 4:1, about 1:1, about 1:4, or any values or ranges between the recited value. The solubilizer can be a vinylpyrrolidone polymer, such as vinyl pyrrolidone homopolymer (or povidone), for example, Povidone K30, Plasdone K29/32 and the alike. In some embodiments, the solubilizer is a vinylpyrrolidone polymer with a nominal K value of about 25-35, such as about 29-32. The K-values assigned to various grades of PVP polymer represent a function of the average molecular weight, the degree of polymerization and the intrinsic viscosity. The K-values are derived from viscosity measurements and are calculated according to Fikentscher's formula. As those skilled in the art would understand that any nominal K value allows certain variations from the nominal value, typically, 90-108%. For example, for Povidone K30, i.e., the nominal K value is 30, the US Pharmacopeia and European Pharmacopeia typically allow a 90%-108% variation of the stated value; thus, a povidone having a K value ranging between 27.0-32.4 is within the specification for Povidone K30 polymers. Unless otherwise obvious from context, the K value referred to herein should be understood as nominal K value. In any of the embodiments described herein, unless otherwise specified or contradictory from context, the vinylpyrrolidone polymer, such as vinyl pyrrolidone homopolymer (or povidone), can have a nominal K value of about 25-35, such as about 29-32. Vinylpyrrolidone polymer as used herein should be understood generally as encompassing both homopolymers and copolymers. The adhesive composition is typically used as an adhesive layer (e.g., drug-in-adhesive layer) in the transdermal delivery device described herein.

In some embodiments, the present disclosure provides a transdermal patch comprising a) a backing layer (e.g., described herein); and b) the adhesive composition or adhesive layer disclosed herein. The adhesive surface is typically protected with a release liner prior to use. Suitable release liners are described herein. In some embodiments, the transdermal patch comprises, consists essentially of, or consists of a) a backing layer; b) the adhesive composition or adhesive layer disclosed herein; and c) an optionally release liner.

In some embodiments, the transdermal patch herein is a single layer transdermal patch comprising a drug-in-adhesive layer, wherein the drug-in-adhesive layer comprises, consists essentially of, or consists of (1) dextromethorphan; (2) a pressure sensitive adhesive; (3) a skin permeation enhancer (e.g., isopropyl myristate); and (4) a solubilizer (e.g., a vinylpyrrolidone polymer, such as vinyl pyrrolidone homopolymer (or povidone), for example, Povidone K30, Plasdone K29/32 and the alike), wherein at least a portion of dextromethorphan is in a solid state dispersed in the pressure sensitive adhesive, wherein the dextromethorphan is in an amount of at least 15%, preferably, about 15% to about 50% (e.g., about 15%, about 20%, about 25%, about 30%, about 35%, about 40%, about 45%, or about 50%, or any values or ranges between the recited values, such as about 20-40%, about 25-35% etc.) by weight; the skin permeation enhancer (e.g., isopropyl myristate) is in an amount of about 6% to about 12% (e.g., about 6%, about 7%, about 8%, about 9%, about 10%, about 11%, about 12%, or any values or ranges between the recited values, such as about 8-12% etc.) by weight; the pressure sensitive adhesive is in an amount of about 20% to about 60% (e.g., about 30%, about 40%, about 45%, about 50%, about 55%, or about 60%, by weight, or any values or ranges between the recited values, such as about 40-60%, about 45-55% etc.) by weight; and the solubilizer is in an amount of about 6% to about 25% (e.g., about 6%, about 8%, about 10%, about 15%, about 20%, or about 25%, or any values or ranges between the recited values, such as about 8-15% or about 6-20% etc.) by weight. In some embodiments, the drug-in-adhesive layer comprises dextromethorphan as the only active ingredient. In some embodiments, the skin permeation enhancer is isopropyl myristate. The pressure sensitive adhesive can be any of those described herein. Typically, the pressure sensitive adhesive is an acrylate copolymer adhesive, e.g., a poly acrylate vinyl acetate copolymer such as those having non-acidic hydroxyl functional groups, for example, described herein such as Duro-Tak 87-2287 adhesive and the alike, manufactured by Henkel Adhesives. In some embodiments, the pressure sensitive adhesive comprises an acrylate copolymer adhesive and an additional adhesive. For example, in some embodiments, the pressure sensitive adhesive can be a mixture of an acrylate copolymer adhesive (e.g., Durotak 87-2287) and a silicone adhesive (e.g., BIO-7-4202) in various ratios (e.g., a weight ratio of acrylate adhesive to silicone adhesive ranging from about 1:20 to about 20:1, e.g., about 10:1 to about 1:10, such as about 10:1, about 4:1, about 1:1, about 1:4, or any values or ranges between the recited value. The solubilizer can typically be a vinylpyrrolidone polymer, such as vinyl pyrrolidone homopolymer (or povidone), for example, Povidone K30, Plasdone K29/32 and the alike. In some embodiments, the solubilizer is a vinylpyrrolidone homopolymer polymer with a nominal K value of about 25-35, such as about 29-32.

In some embodiments, the drug-in-adhesive layer comprises, consists essentially of, or consists of (1) dextromethorphan; (2) a poly acrylate vinyl acetate copolymer pressure sensitive adhesive, such as those having non-acidic hydroxyl functional groups, for example, described herein such as Duro-Tak 87-2287 adhesive and the alike; (3) isopropyl myristate; and (4) a vinylpyrrolidone polymer, such as vinyl pyrrolidone homopolymer (or povidone), for example, Povidone K30, Plasdone K29/32 and the alike, wherein the ranges/amounts of each components can be any of those described herein as suitable in any combination.

The transdermal patch typically has an active surface area of about 30 cm² to about 150 cm², e.g., about 30 cm², about 40 cm², about 50 cm², about 60 cm², about 70 cm², about 80 cm², about 90 cm², about 100 cm², about 120 cm², about 150 cm², or any values or ranges between the recited values, such as about 30-100 cm², about 40-60 cm², about 60-80 cm², etc. In some embodiments, the transdermal patch has an active surface area of about 70 cm². In some embodiments, the transdermal patch can also have an active surface area of greater than about 150 cm², e.g., up to 300 cm².

The transdermal patch herein can also be configured to contain desired amounts of dextromethorphan. In some embodiments, the transdermal patch comprises a drug-in-adhesive layer comprising, consisting essentially of, or consisting of (1) about 150 mg to about 1000 mg of dextromethorphan, e.g., about 200 mg, about 250 mg, about 300 mg, about 350 mg, about 400 mg, about 450 mg, about 500 mg, about 600 mg, about 750 mg, about 900 mg, about 1000 mg, or any values or ranges between the recited values, such as about 250-400 mg, about 300-400 mg, or about 350-450 mg, or about 300-800 mg, about 350-900 mg, etc. of dextromethorphan; (2) about 30 mg to about 350 mg of isopropyl myristate, e.g., about 30 mg, about 50 mg, about 75 mg, about 100 mg, about 125 mg, about 150 mg, about 175 mg, about 200 mg, about 250 mg, about 300 mg, about 350 mg, or any values or ranges between the recited values, such as about 50-150 mg or about 75-125 mg, about 75-300 mg, about 50-250 mg, etc. of isopropyl myristate; (3) about 150 mg to about 1800 mg of a pressure sensitive adhesive, e.g., about 300 mg, about 350 mg, about 400 mg, about 450 mg, about 500 mg, about 550 mg, about 600 mg, about 700 mg, about 800 mg, about 900 mg, about 1000 mg, about 1200 mg, about 1400 mg, about 1600 mg, about 1800 mg, or any values or ranges between the recited values, such as about 350-600 mg, about 400-650 mg, or about 450-700 mg, about 450-1200 mg, about 350-1000 mg, about 400-1600 mg, etc. of the pressure sensitive adhesive; and (4) a solubilizer in an amount of about 30 mg to about 350 mg, e.g., in an amount of e.g., about 30 mg, about 50 mg, about 75 mg, about 100 mg, about 125 mg, about 150 mg, about 175 mg, about 200 mg, about 250 mg, about 300 mg, about 350 mg, or any values or ranges between the recited values, such as about 50-150 mg or about 75-125 mg, about 75-300 mg, about 50-250 mg, etc. At least a portion of dextromethorphan exists in a solid state dispersed in the pressure sensitive adhesive. In some embodiments, the pressure sensitive adhesive is an acrylate based adhesive, e.g., acrylate copolymers. In some embodiments, the pressure sensitive adhesive is a poly acrylate vinyl acetate copolymer such as those having non-acidic hydroxyl functional groups, for example, described herein such as Duro-Tak 87-2287 adhesive and the alike. In some embodiments, the pressure sensitive adhesive comprises an acrylate copolymer adhesive and an additional adhesive. For example, in some embodiments, the pressure sensitive adhesive can be a mixture of an acrylate copolymer adhesive (e.g., Durotak 87-2287) and a silicone adhesive (e.g., BIO-7-4202) in various ratios (e.g., a weight ratio of acrylate adhesive to silicone adhesive ranging from about 1:20 to about 20:1, e.g., about 10:1 to about 1:10, such as about 10:1, about 4:1, about 1:1, about 1:4, or any values or ranges between the recited value. In some embodiments, the solubilizer is a vinylpyrrolidone polymer, such as vinyl pyrrolidone homopolymer (or povidone), for example, Povidone K30, Plasdone K29/32 and the alike. In some embodiments, the solubilizer is a vinylpyrrolidone homopolymer polymer with a nominal K value of about 25-35, such as about 29-32. Typically, the drug-in-adhesive layer comprises dextromethorphan in an amount of about 20% to about 40% (e.g., described herein, such as about 30%) by weight; isopropyl myristate in an amount of about 6% to about 12% (e.g., described herein, such as about 10%) by weight; the pressure sensitive adhesive in an amount of about 40% to about 60% (e.g., described herein, such as about 50%) by weight; and the solubilizer in an amount of about 6% to about 20% (e.g., described herein, such as about 10%) by weight. In some embodiments, the drug-in-adhesive layer comprises dextromethorphan as the only active ingredient. The transdermal patch typically has an active surface area of about 30 cm² to about 150 cm², e.g., about 30 cm², about 40 cm², about 50 cm², about 60 cm², about 70 cm², about 80 cm², about 90 cm², about 100 cm², about 150 cm², or any values or ranges between the recited values, such as about 40-60 cm², about 60-80 cm², etc. In some embodiments, the transdermal patch has an active surface area of about 70 cm². In some embodiments, the transdermal patch can also have an active surface area of greater than about 150 cm², e.g., up to 300 cm². In any of the embodiments described herein, the transdermal patch can be in the form of a single layer patch unless otherwise obviously contradictory from context.

The transdermal patch herein typically has a total dextromethorphan loading of about 2 mg/cm² to about 25 mg/cm², such as about 2.5 mg/cm², about 3 mg/cm², about 4 mg/cm², about 5 mg/cm², about 6 mg/cm², about 7 mg/cm², about 8 mg/cm², about 9 mg/cm², about 10 mg/cm², about 12 mg/cm², about 15 mg/cm², about 20 mg/cm², about 25 mg/cm², or any values or ranges between the recited values, such as about 3-10 mg/cm², about 4-7 mg/cm², about 5-12 mg/cm², about 8-25 mg/cm², etc.

Typically, the amount of dextromethorphan included in the transdermal patch herein is sufficient to deliver a therapeutically effective amount of dextromethorphan to a subject in need thereof. In some embodiments, the amount of dextromethorphan included in the transdermal patch herein is sufficient to transdermally deliver a daily dose of about 15 mg to about 50 mg (e.g., about 15 mg, about 20 mg, about 30 mg, about 40 mg, about 50 mg, or any values or ranges between the recited values, such as about 20-50 mg, about 30-50 mg, or about 20-40 mg, etc.) of dextromethorphan to a subject in need thereof, for at least 7 days. Preferably, one single patch is used to deliver the daily dose herein.

The transdermal patches herein typically have a dextromethorphan flux suitable for delivering a therapeutically effective amount to a subject in need thereof. For example, in some embodiments, the transdermal patch has a dextromethorphan flux of at least about 200 ug/cm²/day for at least seven days, when measured in vitro using human cadaver skin, such as about 200 ug/cm²/day, about 300 ug/cm²/day, about 400 ug/cm²/day, about 500 ug/cm²/day, about 600 ug/cm²/day, about 700 ug/cm²/day, about 800 ug/cm²/day, about 1000 ug/cm²/day, or any values or ranges between the recited values, such as about 200-800 ug/cm²/day, about 300-800 ug/cm²/day, about 400-800 ug/cm²/day, about 500-800 ug/cm²/day, etc., for seven days or more. In some embodiments, during the patch-on period, the transdermal patch can transdermally deliver dextromethorphan to the human subject at a substantially same rate, for example, the cumulative daily permeation of dextromethorphan does not vary from the average cumulative daily permeation of dextromethorphan by more than 350 ug/cm²/day, preferably, does not vary by more than 250 ug/cm²/day, more preferably, does not vary by more than 200 ug/cm²/day. As discussed in WO 2021/202329 A1, it was found that the use of vinylpyrrolidone polymer (Plasdone K29/32) as a crystallization inhibitor can significantly enhance the flux of dextromethorphan from the transdermal patches herein, both in vitro and in vivo. Unexpectedly, the use of vinylpyrrolidone polymer (Plasdone K29/32), although not to prevent dextromethorphan crystallization, allows the transdermal patches herein to have a desirable dextromethorphan permeation even when some of the dextromethorphan exists in a solid state, which is generally considered as impeding permeation of drugs from transdermal patches.

In some embodiments, the present disclosure also provides a method of selecting a transdermal patch for the methods herein (e.g., methods of treating PBA), the method comprises measuring in vitro dextromethorphan flux from a transdermal patch disclosed herein (e.g., those shown in [1]-[51] in the Brief Summary Section), e.g., using human cadaver skin, and selecting a transdermal patch having dextromethorphan flux of at least about 200 ug/cm²/day for at least seven days, when measured in vitro using human cadaver skin, such as about 200 ug/cm²/day, about 300 ug/cm²/day, about 400 ug/cm²/day, about 500 ug/cm²/day, about 600 ug/cm²/day, about 700 ug/cm²/day, about 800 ug/cm²/day, about 1000 ug/cm²/day, or any values or ranges between the recited values, such as about 200-800 ug/cm²/day, about 300-800 ug/cm²/day, about 400-800 ug/cm²/day, about 500-800 ug/cm²/day, etc., for seven days or more.

In some specific embodiments, the transdermal patch comprises, consists essentially of, or consists of a) a backing layer (e.g., described herein), b) a drug-in-adhesive layer, and c) an optional release liner, wherein the drug-in-adhesive layer comprises (1) dextromethorphan in an amount of about 15% to about 50% (e.g., described herein, such as about 30%) by weight; (2) a poly acrylate vinyl acetate copolymer pressure sensitive adhesive, such as those having non-acidic hydroxyl functional groups, for example, described herein such as Duro-Tak 87-2287 adhesive and the alike, in an amount of about 20% to about 60% (e.g., described herein, such as about 50%) by weight; (3) isopropyl myristate in an amount of about 6% to about 12% (e.g., described herein, such as about 10%) by weight; and (4) a vinylpyrrolidone polymer, such as vinyl pyrrolidone homopolymer (or povidone), for example, Povidone K30, Plasdone K29/32 and the like, in an amount of about 6% to about 20% (e.g., described herein, such as about 10%) by weight. In some embodiments, the transdermal patch has at least a portion of the dextromethorphan in a solid state. In some embodiments, the transdermal patch has a dextromethorphan flux of at least about 300 ug/cm²/day (e.g., about 300 ug/cm²/day to about 650 ug/cm²/day) for 7 days or more, when measured in vitro using human cadaver skin.

In some specific embodiments, the present disclosure provides a monolithic transdermal patch, which comprises, consists essentially of, or consists of a) a backing layer (e.g., described herein), b) a drug-in-adhesive layer, and c) an optional release liner, wherein the drug-in-adhesive layer comprises, consists essentially of, or consists of (1) about 150 mg to about 1000 mg (e.g., described herein) of dextromethorphan; (2) about 150 mg to about 1800 mg (e.g., described herein) a poly acrylate vinyl acetate copolymer pressure sensitive adhesive, such as those having non-acidic hydroxyl functional groups, for example, described herein such as Duro-Tak 87-2287 adhesive and the alike; (3) about 30 mg to about 350 mg (e.g., described herein) of isopropyl myristate; and (4) about 30 mg to about 350 mg (e.g., described herein) of a vinylpyrrolidone polymer, such as vinyl pyrrolidone homopolymer (or povidone), for example, Povidone K30, Plasdone K29/32 and the like. In some embodiments, the transdermal patch has at least a portion of the dextromethorphan in a solid state. In some embodiments, the weigh percentage of ingredients in the drug-in-adhesive layer can be (1) dextromethorphan in an amount of about 15% to about 50% (e.g., described herein, such as about 30%) by weight; (2) the poly acrylate vinyl acetate copolymer pressure sensitive adhesive, such as those having non-acidic hydroxyl functional groups, for example, described herein such as Duro-Tak 87-2287 adhesive and the alike, in an amount of about 20% to about 60% (e.g., described herein, such as about 50%) by weight; (3) isopropyl myristate in an amount of about 6% to about 12% (e.g., described herein, such as about 10%) by weight; and (4) the vinylpyrrolidone polymer, such as vinyl pyrrolidone homopolymer (or povidone), for example, Povidone K30, Plasdone K29/32 and the like, in an amount of about 6% to about 20% (e.g., described herein, such as about 10%) by weight. In some embodiments, the transdermal patch has an active surface area of about 30 cm² to about 150 cm² (e.g., described herein, such as about 70 cm²). In some embodiments, the transdermal patch has a dextromethorphan flux of at least about 300 ug/cm²/day for at least 7 days (e.g., about 300 ug/cm²/day to about 650 ug/cm²/day) when measured in vitro using human cadaver skin.

The transdermal patches and formulations are preferably storage stable when stored at room temperature (25±2 °C) at 60% relative humidity (RH)+5% RH for about 1 month, 3 months, 6 months or longer. By storage stable, it is to be meant that the transdermal patches or formulations would be accepted by those skilled in the art as equivalent to the initial transdermal patches or formulations, i.e., at the beginning of the storage. Storage stable is typically characterized by one or more of the following: (1) substantially same amount of drug related impurities, no significant increased amount of either individual or total impurities; (2) substantially same amount of dextromethorphan; (3) substantially same physical properties such as peel adhesion, shear adhesion, task force, release force, etc.; and (4) substantially same drug release rate and/or dextromethorphan permeation rate. “Substantially same” should be understood as meaning within 80-125% or measurement error margin. For example, patches prepared according to Examples 1 and 2 herein were found to be storage stable after storage at room temperature (25±2 °C) at 60% relative humidity (RH)±5% RH or at 40 °C and 75% RH for at least 1 month.

In some embodiments, the present disclosure also provides a method of preparing a transdermal delivery device or adhesive composition. In some embodiments, the method comprises: a) mixing dextromethorphan, an adhesive (e.g., a pressure sensitive adhesive described herein such as Duro-Tak 87-2287), a permeation enhancer (e.g., isopropyl myristate) and a solubilizer (e.g., described herein such as a vinylpyrrolidone polymer, such as vinyl pyrrolidone homopolymer (or povidone), for example, Povidone K30, Plasdone K29/32 and the alike) in a suitable solvent (e.g., an organic solvent such as an ester solvent or an alcohol solvent, typically volatile, e.g., ethyl acetate or isopropanol or combinations thereof) to form a homogenous mixture; b) casting the homogenous mixture onto a release liner; and c) drying the casting to remove the solvent to form an adhesive composition on the release liner. In some embodiments, dextromethorphan and the adhesive (e.g., a pressure sensitive adhesive described herein such as Duro-Tak 87-2287) are mixed separately and then mixed with the remaining ingredients. In some embodiments, the permeation enhancer and the solubilizer are mixed separately and then mixed with a mixture of dextromethorphan and the adhesive (e.g., a pressure sensitive adhesive described herein such as Duro-Tak 87-2287). In some embodiments, the method further comprises laminating the adhesive composition to a backing layer. Suitable amount of dextromethorphan and suitable adhesive, solubilizer, permeation enhancer and their respective amount, can include any of those described herein in any combination. The adhesive composition, with or without the release liner, and transdermal delivery device prepared by the methods herein are also novel aspects of the present disclosure. Some exemplary procedures are described herein in the Examples section.

TDD with a Reservoir Layer

In some embodiments, a reservoir layer is included in the transdermal delivery device herein. For example, for high daily doses and/or application for an extended period of time (e.g., 7 days or more), the reservoir layer can provide more sustained flux of dextromethorphan to a user.

In some embodiments, the transdermal delivery device comprises an adhesive layer comprising an adhesive and a reservoir layer comprising dextromethorphan. In some embodiments, the adhesive layer comprises dextromethorphan dispersed in the adhesive. In some embodiments, the adhesive layer does not include dextromethorphan, other than through equilibrium with the reservoir layer. In some embodiments, the adhesive layer comprises dextromethorphan dispersed in the adhesive. In some embodiments, the reservoir layer comprises dextromethorphan in an adhesive. In some embodiments, the reservoir layer is sandwiched between the adhesive layer and a backing layer, and the adhesive layer is a skin-contacting layer (preferably a skin-contacting drug-in-adhesive layer). In some embodiments, the reservoir layer can be sandwiched between two adhesive layers which can be the same or different. For example, in some embodiments, the two adhesive layers can have the same ingredients with the same concentrations, and in some embodiments, can also have the same thickness. However, in some embodiments, the two adhesive layers can have different ingredients, or same ingredients with different concentrations, or have different thickness, etc.

In some embodiments, the reservoir layer is separated from the adhesive layer by a membrane, e.g., a rate controlling membrane such as a microporous membrane. The reservoir layer preferably contains an adhesive; however, other designs of the reservoir layer are also suitable when compatible with the adhesive layer and the backing layer. For example, in some embodiments, the reservoir layer can be a scrim/nonwoven fabric saturated with dextromethorphan, or having dextromethorphan dispersed in other suitable carrier/substrate.

Dextromethorphan can be included in the adhesive layer and reservoir layer in various concentrations. Typically, the concentration of dextromethorphan in the reservoir layer is higher than that in the adhesive layer. For example, in some embodiments, the adhesive layer can comprise dextromethorphan in an amount of about 2% to about 12% (e.g., about 2%, about 4%, about 6%, about 8%, about 10%, about 12%, or any range between the recited values) by weight of the adhesive layer; whereas the reservoir layer can comprise dextromethorphan in an amount of about 20% or more, for example, about 30% or more, about 40% or more, about 50% or more, such as about 20% to about 60%, about 30% to about 50%, by weight of the reservoir layer. In some embodiments, the adhesive layer comprises dextromethorphan in an amount of about 6% to about 12% (e.g., about 6%, about 7%, about 8%, about 9%, about 10%, about 11%, about 12%, or any values or ranges between the recited values) by weight of the adhesive layer. In some embodiments, the adhesive layer comprises dextromethorphan at or near the saturation concentration in the adhesive, for example, about 10% by weight in an acrylate adhesive. In some embodiments, the reservoir layer comprises dextromethorphan above the saturation concentration in the adhesive. In other words, the dextromethorphan in the reservoir layer is oversaturated and can therefore contain solid dextromethorphan, which can serve as a drug depot.

Suitable adhesives for the adhesive layer and the reservoir layer, as applicable, include any of those described herein, preferably pressure sensitive adhesives. The adhesives included in the adhesive layer and reservoir layer can be the same or different. In some embodiments, the adhesives included in the adhesive layer and reservoir layer are the same, for example, acrylate based adhesives. Other suitable adhesives include a polyisobutylene adhesive, a silicone polymer adhesive, an acrylate copolymer adhesive (e.g., a poly acrylate vinyl acetate copolymer, such as those having non-acidic hydroxyl functional groups, for example, described herein such as Duro-Tak 87-2287 adhesive and the alike), or a combination thereof. For example, in any of the embodiments described herein, unless directly contrary from context, the pressure sensitive adhesive can be a poly acrylate vinyl acetate copolymer, e.g., containing non-acidic hydroxyl functional groups, such as DuroTak® 2287 adhesives as described herein. In some embodiments, the adhesive can be a mixture of an acrylate copolymer adhesive (e.g., Durotak 87-2287) and a silicone adhesive (e.g., BIO-7-4202) in various ratios (e.g., a weight ratio of acrylate adhesive to silicone adhesive ranging from about 1:20 to about 20:1). In some embodiments, the weight ratio of the acrylate adhesive to silicon adhesive ranges from about 10:1 to about 1:10 (e.g., about 10:1, about 4:1, about 1:1, about 1:4, or any values or ranges between the recited value). In any of the embodiments described herein, the adhesive layer can be configured for adhering to a user's skin continuous for at least 7 days.

In some embodiments, the adhesive layer comprises a pressure sensitive adhesive in an amount of about 50% to about 90% by weight of the adhesive layer. In some embodiments, the pressure sensitive adhesive is present in an amount of about 60% to about 85% (e.g., about 60%, about 70%, about 75%, about 80%, about 85%, or any values or ranges between the recited values) by weight of the adhesive layer. In some embodiments, the reservoir layer can include a pressure sensitive adhesive in an amount of about 20% to about 60% by weight of the reservoir layer. For example, in some embodiments, the pressure sensitive adhesive is present in an amount of about 20% to about 50% (e.g., about 20%, about 30%, about 35%, about 40%, about 50%, or any values or ranges between the recited values) by weight of the reservoir layer.

Suitable sizes for the transdermal delivery device are described herein. In some embodiments, the transdermal delivery device has an active surface area of about 5 cm² to about 200 cm². In some embodiments, the transdermal delivery device has an active surface area of about 10 cm² to about 150 cm². In some embodiments, the transdermal delivery device has an active surface area of about 30 cm² to about 150 cm² (e.g., about 30 cm², about 40 cm², about 50 cm², about 60 cm², about 70 cm², about 80 cm², about 90 cm², about 100 cm², about 120 cm², about 150 cm², or any values or ranges between the recited values).

The adhesive layer and reservoir layer can be of various thickness. For example, in some embodiments, the adhesive layer is about 0.1 mil to about 10 mils thick (e.g., about 0.5 mil to about 10 mils, about 1 mil to 10 mils). In some embodiments, the reservoir layer can also be about 0.1 mil to about 10 mils thick (e.g., about 0.5 mil to about 10 mils, about 1 mil to 10 mils). For example, in preferred embodiments, the adhesive layer is about 0.4 mil to about 3 mils thick. In some preferred embodiments, the adhesive layer is about 0.4 mil to about 5 mils thick. In some embodiments, the reservoir layer is about 2 mils to about 7.5 mils such as about 4 mils. In some embodiments, the reservoir layer has a thickness of at least about 100%, preferably, about 100-500% (preferably, 100-600% or 100-300%) of that of the adhesive layer. In some embodiments, the amount of dextromethorphan in the reservoir layer is at least about 200%, preferably, about 200-3000% (preferably, about 300-1500%, such as about 300-800% or about 400-600%) of that in the adhesive layer. In some embodiments, the total weight of the reservoir layer is at least about 100%, preferably, about 100-800% (preferably, 100-600% or 120-300%) of that of the adhesive layer.

Skin permeation enhancers can also be included in the adhesive layer and the reservoir layer. For example, in some embodiments, the adhesive layer comprises a skin permeation enhancer selected from isopropyl myristate, oleyl oleate, oleic acid, glycerol monooleate, other fatty acids and fatty acid esters with carbon chain lengths of C₁₂ to C₁₈, and combinations thereof. In some embodiments, the adhesive layer comprises isopropyl myristate. Similarly, in some embodiments, the reservoir layer comprises a skin permeation enhancer selected from isopropyl myristate, oleyl oleate, oleic acid, glycerol monooleate, other fatty acids and fatty acid esters with carbon chain lengths of C₁₂ to C₁₈, and combinations thereof. In some embodiments, the reservoir layer comprises isopropyl myristate.

Various amounts of skin permeation enhancers can be used for the adhesive layer and the reservoir layer. Typically, the skin permeation enhancer can be present in an amount of about 2% to about 15% by weight of the adhesive layer or reservoir layer. For example, in some embodiments, the skin permeation enhancer is present in an amount of about 6% to about 12% (e.g., about 6%, about 7%, about 8%, about 9%, about 10%, about 11%, about 12%, or any values or ranges between the recited values) by weight of the adhesive layer. In some embodiments, the skin permeation enhancer is present in an amount of about 6% to about 12% (e.g., about 6%, about 7%, about 8%, about 9%, about 10%, about 11%, about 12%, or any values or ranges between the recited values) by weight of the reservoir layer.

In some embodiments, the adhesive layer can include a crystallization inhibitor, such as a vinylpyrrolidone polymer, such as vinyl pyrrolidone homopolymer (or povidone), for example, Povidone K30, Plasdone K29/32 and the alike. In some embodiments, the crystallization inhibitor can be present in an amount of about 6% to about 12% (e.g., about 6%, about 7%, about 8%, about 9%, about 10%, about 11%, about 12%, or any values or ranges between the recited values, such as about 6-12% etc.) by weight. In some embodiments, the adhesive layer does not include a crystallization inhibitor.

In some embodiments, the reservoir layer can include a solubilizer, such as a vinylpyrrolidone polymer, such as vinyl pyrrolidone homopolymer (or povidone), for example, Povidone K30, Plasdone K29/32 and the alike. In some embodiments, the solubilizer is present in an amount of about 6%, about 8%, about 10%, about 15%, about 20%, or about 25%, or any values or ranges between the recited values, such as about 15-25% or about 10-20% etc., of the reservoir layer. In some embodiments, the reservoir layer does not include a solubilizer.

It should be noted that the identities of ingredients such as adhesives, skin permeation enhancers, agents, and amounts thereof, for the adhesive layer and the reservoir layer are independently selected, which can be the same or different. Typically, the amounts can vary whereas the identity can be the same.

### Exemplary Transdermal Delivery Devices Having a Reservoir Layer

In some embodiments, the present disclosure also provides the following non-limiting exemplary transdermal patches having an adhesive layer and a reservoir layer and related transdermal formulations.

In some embodiments, the transdermal patch comprises (1) a backing layer; (2) a skin-contacting drug-in-adhesive layer (e.g., any of those described herein); and (3) a reservoir layer. In some embodiments, the transdermal patch can be characterized as having a bilayer structure, or a bilayer batch, in which the reservoir layer is sandwiched by the backing layer and the skin-contacting drug-in-adhesive layer. To be clear, the skin contacting drug-in-adhesive layer refers to the drug-in-adhesive layer in the transdermal patch, when applied to the skin of a subject user, contacts the skin. The skin contacting drug-in-adhesive layer is typically protected by a release liner prior to use. In some embodiments, the transdermal patch consists of the backing layer, skin contacting drug-in-adhesive layer, reservoir layer, and optionally a release liner.

In some embodiments, the skin-contacting drug-in-adhesive layer comprises 1) dextromethorphan in an amount of about 6% to about 12% by weight; 2) isopropyl myristate in an amount of about 6% to about 12% by weight; 3) a first pressure sensitive adhesive, preferably, an acrylate based pressure sensitive adhesive, in an amount of about 65% to about 85% by weight; and 4) a crystallization inhibitor in an amount of about 6% to about 12% by weight; and the reservoir layer comprises 1) dextromethorphan in an amount of at least 15% by weight; 2) isopropyl myristate in an amount of about 6% to about 12% by weight; (3) a second pressure sensitive adhesive in an amount of about 20% to about 60% by weight; and (4) a solubilizer in an amount of about 6% to about 25% by weight, wherein at least a portion of the dextromethorphan in the reservoir layer is in a solid state dispersed in the second pressure sensitive adhesive. In some alternative embodiments, the reservoir layer does not include a solubilizer and/or the skin-contacting drug-in-adhesive layer does not include a crystallization inhibitor.

The first and second pressure sensitive adhesive are the same or different, preferably the same. Typically, the first and second pressure sensitive adhesive are both acrylate based pressure sensitive adhesive. In some embodiments, the first pressure sensitive adhesive is an acrylate copolymer adhesive, e.g., a poly acrylate vinyl acetate copolymer pressure sensitive adhesive, such as those having non-acidic hydroxyl functional groups, for example, described herein such as Duro-Tak 87-2287 adhesive and the alike. In some embodiments, the second pressure sensitive adhesive is an acrylate copolymer adhesive, e.g., a poly acrylate vinyl acetate copolymer pressure sensitive adhesive, such as those having non-acidic hydroxyl functional groups, for example, described herein such as Duro-Tak 87-2287 adhesive and the alike.

The first pressure sensitive adhesive is typically present in the skin-contacting drug-in-adhesive layer in an amount of about 65%, about 70%, about 75%, about 80%, or about 85%, by weight, or any values or ranges between the recited values, such as about 65-75%, about 70-85% etc., of the skin-contacting drug-in-adhesive layer.

The second pressure sensitive adhesive is typically present in the reservoir layer in an amount of about 20%, about 30%, about 35%, about 40%, about 45%, about 50%, or about 60%, by weight, or any values or ranges between the recited values, such as about 35-45%, about 30-50%, etc. of the reservoir layer.

The isopropyl myristate is typically present in an amount of about 6%, about 7%, about 8%, about 9%, about 10%, about 11%, or about 12%, by weight, or any values or ranges between the recited values, such as about 8-12% etc., of the skin-contacting drug-in-adhesive layer. The concentration of isopropyl myristate in the reservoir layer is typically similar to that of the skin-contacting drug-in-adhesive layer. For example, in some embodiments, the reservoir layer also comprises isopropyl myristate in an amount of about 6%, about 7%, about 8%, about 9%, about 10%, about 11%, or about 12%, by weight, or any values or ranges between the recited values, such as about 8-12% etc., of the reservoir layer.

The dextromethorphan in the skin-contacting drug-in-adhesive layer is typically below its saturation concentration in the adhesive layer, in other words, the skin-contacting drug-in-adhesive layer typically does not contain dextromethorphan in a solid state. In some embodiments, the dextromethorphan in the skin-contacting drug-in-adhesive layer is at or close to its saturation concentration in the adhesive layer. In some embodiments, the dextromethorphan in the skin-contacting drug-in-adhesive layer is in an amount of about 6%, about 7%, about 8%, about 9%, about 10%, about 11%, or about 12%, by weight, or any values or ranges between the recited values, such as about 6-10% etc., of the skin-contacting drug-in-adhesive layer.

In some embodiments, the skin-contacting drug-in-adhesive layer comprises the crystallization inhibitor in an effective amount to prevent dextromethorphan solid formation in the skin-contacting drug-in-adhesive layer. In some embodiments, the crystallization inhibitor is a vinylpyrrolidone polymer, such as vinyl pyrrolidone homopolymer (or povidone), for example, Povidone K30, Plasdone K29/32 and the alike. In some embodiments, the crystallization inhibitor is present in an amount of about 6%, about 7%, about 8%, about 9%, about 10%, about 11%, or about 12%, or any values or ranges between the recited values, such as about 6-12% or 8-12% etc., of the skin-contacting drug-in-adhesive layer.

The dextromethorphan in the reservoir layer is typically above its saturation concentration in the reservoir layer, and at least a portion of the dextromethorphan is in a solid state. In some embodiments, the dextromethorphan in the reservoir layer is in an amount of about 15%, about 20%, about 25%, about 30%, about 35%, about 40%, about 45%, or about 50%, by weight, or any values or ranges between the recited values, such as about 20-40%, about 25-35% etc., of the reservoir layer.

The solubilizer in the reservoir layer is typically a vinylpyrrolidone polymer, such as vinyl pyrrolidone homopolymer (or povidone), for example, Povidone K30, Plasdone K29/32 and the alike. The amount of the solubilizer is not particularly limited, and is typically in an amount of about 6%, about 8%, about 10%, about 15%, about 20%, or about 25%, or any values or ranges between the recited values, such as about 15-25% or about 10-20% etc., of the reservoir layer.

The transdermal patches herein can also be characterized by the different configurations of the skin-contacting drug-in-adhesive layer and reservoir layer, which is believed to contribute to the desired permeation of dextromethorphan for an extended period of time, such as for 7 days or more.

For example, in some embodiments, the dextromethorphan in the reservoir layer can have a concentration that is about 200-800% (preferably, about 300-500%) of that in the skin contacting drug-in-adhesive layer. For example, in some embodiments, the dextromethorphan can have a concentration of about 8% by weight in the skin contacting drug-in-adhesive layer, but the reservoir layer can have a concentration of dextromethorphan of about 30% by weight, i.e., about 3.75 times or 375% of that in the skin contacting drug-in-adhesive layer.

In some embodiments, the amount of dextromethorphan in the reservoir layer can be at least about 200%, preferably, about 200-3000% (preferably, about 300-1500%, such as about 300-800% or about 400-600%) of that in the skin contacting drug-in-adhesive layer. For example, in some embodiments, the skin contacting drug-in-adhesive layer can have about 56 mg of dextromethorphan but the reservoir layer can have about 120 mg to about 900 mg or more of dextromethorphan. In some embodiments, the ratio of the amount of dextromethorphan of the reservoir layer to the amount of dextromethorphan of the skin contacting drug-in-adhesive layer ranges from about 2:1 to about 30:1, such as about 3:1, about 4:1, about 5:1, about 8:1, about 10:1, about 15:1, about 20:1, about 30:1, or any values or ranges between the recited values.

Typically, the skin contacting drug-in-adhesive layer is thinner than the reservoir layer. In some embodiments, the skin contacting drug-in-adhesive layer has a thickness of about 0.4 mil to about 3 mils. In some embodiments, the skin contacting drug-in-adhesive layer has a thickness of about 0.4 mil to about 5 mils. In some embodiments, the reservoir layer has a thickness of about 2 mils to about 7.5 mils such as about 4 mils. In some embodiments, the reservoir layer has a thickness of at least about 100%, preferably, about 100-800% (preferably, 100-600% or 120-300%) of that of the skin contacting drug-in-adhesive layer. In some embodiments, the total weight of the reservoir layer is at least about 100%, preferably, about 100-800% (preferably, 100-600% or 120-300%) of that of the skin contacting drug-in-adhesive layer. In some embodiments, the ratio of the thickness of the reservoir layer to the thickness of the skin contacting drug-in-adhesive layer ranges from about 1:1 to about 10:1, such as about 1:1, about 1.3:1, about 1.5:1, about 2:1, about 3:1, about 5:1, about 8:1, about 10:1, or any values or ranges between the recited values. In some embodiments, the ratio of the total weight of the reservoir layer to the total weight of the skin contacting drug-in-adhesive layer ranges from about 1:1 to about 10:1, such as about 1:1, about 1.3:1, about 1.5:1, about 2:1, about 3:1, about 5:1, about 8:1, about 10:1, or any values or ranges between the recited values.

The total amount of dextromethorphan in the transdermal patch herein, i.e., including both dextromethorphan in the skin contacting drug-in-adhesive layer and reservoir layer, is typically in the range of about 150 mg to about 1000 mg, e.g., about 200 mg, about 250 mg, about 300 mg, about 350 mg, about 400 mg, about 450 mg, about 500 mg, about 600 mg, about 750 mg, about 900 mg, about 1000 mg, or any values or ranges between the recited values, such as about 250-400 mg, about 300-400 mg, or about 350-450 mg, or about 300-800 mg, about 350-900 mg, etc. In some embodiments, the transdermal patch herein has a total dextromethorphan loading of about 2 mg/cm² to about 25 mg/cm², such as about 2.5 mg/cm², about 3 mg/cm², about 4 mg/cm², about 5 mg/cm², about 6 mg/cm², about 7 mg/cm², about 8 mg/cm², about 9 mg/cm², about 10 mg/cm², about 12 mg/cm², about 15 mg/cm², about 20 mg/cm², about 25 mg/cm², or any values or ranges between the recited values, such as about 3-10 mg/cm², about 4-7 mg/cm², about 5-12 mg/cm², about 8-25 mg/cm², etc.

The total amount of isopropyl myristate in the transdermal patch herein, i.e., including both isopropyl myristate in the skin contacting drug-in-adhesive layer and reservoir layer, is typically in the range of about 30 mg to about 350 mg, e.g., about 30 mg, about 50 mg, about 75 mg, about 100 mg, about 125 mg, about 150 mg, about 175 mg, about 200 mg, about 250 mg, about 300 mg, about 350 mg, or any values or ranges between the recited values, such as about 50-150 mg or about 75-125 mg, about 75-300 mg, about 50-250 mg, etc.

The combined amount of the first and second pressure sensitive adhesive is typically in the range of about 150 mg to about 1800 mg, e.g., about 300 mg, about 350 mg, about 400 mg, about 450 mg, about 500 mg, about 550 mg, about 600 mg, about 700 mg, about 800 mg, about 900 mg, about 1000 mg, about 1200 mg, about 1400 mg, about 1600 mg, about 1800 mg, or any values or ranges between the recited values, such as about 350-600 mg, about 400-650 mg, or about 450-700 mg, about 450-1200 mg, about 350-1000 mg, about 400-1600 mg, etc.

Preferably, the transdermal patch has an active surface area of about 30 cm² to about 150 cm². In some embodiments, the transdermal patch typically has an active surface area of about 30 cm² to about 150 cm², e.g., about 30 cm², about 40 cm², about 50 cm², about 60 cm², about 70 cm², about 80 cm², about 90 cm², about 100 cm², about 120 cm², about 150 cm², or any values or ranges between the recited values, such as about 30-100 cm², about 40-60 cm², about 60-80 cm², etc. In some embodiments, the transdermal patch has an active surface area of about 70 cm². In some embodiments, the transdermal patch can also have an active surface area of greater than about 150 cm², e.g., up to 300 cm².

Typically, the amount of dextromethorphan included in the transdermal patch herein, such as a bilayer patch herein, is sufficient to deliver a therapeutically effective amount of dextromethorphan to a subject in need thereof. In some embodiments, the amount of dextromethorphan included in the transdermal patch herein is sufficient to transdermally deliver a daily dose of about 15 mg to about 50 mg (e.g., about 15 mg, about 20 mg, about 30 mg, about 40 mg, about 50 mg, or any values or ranges between the recited values, such as about 20-50 mg, about 30-50 mg, or about 20-40 mg, etc.) of dextromethorphan to a subject in need thereof, for at least 7 days. Preferably, one single patch is used to deliver the daily dose herein.

The transdermal patches herein typically have a dextromethorphan flux suitable for delivering a therapeutically effective amount to a subject in need thereof. Unexpectedly, the use of vinylpyrrolidone polymer (Plasdone K29/32) in both the skin containing drug-in-adhesive layer and the reservoir layer in patches having a reservoir layer, such as a bilayer patch herein, allows the transdermal patches herein to have a desirable dextromethorphan permeation even when some of the dextromethorphan exists in a solid state in the reservoir layer, which is generally considered as impeding permeation of drugs from transdermal patches. For example, in some embodiments, the transdermal patch, such as a bilayer patch herein, has a dextromethorphan flux of at least about 200 ug/cm²/day for at least seven days, when measured in vitro using human cadaver skin, such as about 200 ug/cm²/day, about 300 ug/cm²/day, about 400 ug/cm²/day, about 500 ug/cm²/day, about 600 ug/cm²/day, about 700 ug/cm²/day, about 800 ug/cm²/day, about 1000 ug/cm²/day, or any values or ranges between the recited values, such as about 200-800 ug/cm²/day, about 300-800 ug/cm²/day, about 400-800 ug/cm²/day, about 500-800 ug/cm²/day, etc., for seven days or more. In some embodiments, the transdermal patch has a dextromethorphan flux of at least about 300 ug/cm²/day (e.g., about 300 ug/cm²/day to about 650 ug/cm²/day) for 7 days or more, when measured in vitro using human cadaver skin.

The transdermal patches having a reservoir layer are preferably storage stable when stored at room temperature (25±2 °C) at 60% relative humidity (RH)+5% RH for about 1 month, 3 months, 6 months or longer.

In some embodiments, the present disclosure also provides a method of preparing a transdermal delivery device having a reservoir layer. In some embodiments, the method comprises: 1) preparing a skin contacting drug-in-adhesive layer; 2) preparing a reservoir layer; and 3) laminating the skin contacting drug-in-adhesive layer to the reservoir layer. Typically, the reservoir layer is sandwiched by the skin contacting drug-in-adhesive layer and a backing layer. The skin contacting drug-in-adhesive layer and reservoir layer can be prepared according to the methods herein. For example, the reservoir layer can be prepared by a process comprising a) mixing dextromethorphan, an adhesive (e.g., a pressure sensitive adhesive described herein such as Duro-Tak 87-2287), a permeation enhancer (e.g., isopropyl myristate) and a solubilizer (e.g., described herein such as a vinylpyrrolidone polymer, such as vinyl pyrrolidone homopolymer (or povidone), for example, Povidone K30, Plasdone K29/32 and the alike) in a suitable solvent (e.g., an organic solvent such as an ester solvent or an alcohol solvent, typically volatile, e.g., ethyl acetate or isopropanol or combinations thereof) to form a homogenous mixture; b) casting the homogenous mixture onto a release liner; and c) drying the casting to remove the solvent to form a reservoir adhesive composition on the release liner. Similarly, the skin contacting drug-in-adhesive layer can be prepared by a process comprising a) mixing dextromethorphan, an adhesive (e.g., a pressure sensitive adhesive described herein such as Duro-Tak 87-2287), a permeation enhancer (e.g., isopropyl myristate) and a crystallization inhibitor (e.g., described herein such as a vinylpyrrolidone polymer, such as vinyl pyrrolidone homopolymer (or povidone), for example, Povidone K30, Plasdone K29/32 and the alike) in a suitable solvent (e.g., an organic solvent such as an ester solvent or an alcohol solvent, typically volatile, e.g., ethyl acetate or isopropanol or combinations thereof) to form a homogenous mixture; b) casting the homogenous mixture onto a release liner; and c) drying the casting to remove the solvent to form a skin contacting drug-in-adhesive adhesive composition on the release liner. In some embodiments, dextromethorphan and the adhesive (e.g., a pressure sensitive adhesive described herein such as Duro-Tak 87-2287) are mixed separately and then mixed with the remaining ingredients. In some embodiments, the permeation enhancer and the solubilizer or the crystallization inhibitor are mixed separately and then mixed with a mixture of dextromethorphan and the adhesive (e.g., a pressure sensitive adhesive described herein such as Duro-Tak 87-2287). In some embodiments, the method further comprises laminating the reservoir adhesive composition to a backing layer. Suitable amount of dextromethorphan and suitable adhesive, solubilizer, crystallization inhibitor, permeation enhancer and their respective amounts, can include any of those described herein in any combination. The reservoir adhesive composition and skin contacting drug-in-adhesive adhesive composition, with or without the release liner, and transdermal delivery device prepared by the methods herein are also novel aspects of the present disclosure. Some exemplary procedures are described herein in the Examples section.

Transdermal delivery devices with a flux characteristic described herein can be prepared by those skilled in the art in view of the present disclosure. Preparations of a few transdermal delivery devices are also exemplified in the Examples section. The cumulative drug (dextromethorphan, deuterated dextromethorphan, or a combination thereof) permeated can be adjusted, for example, by varying the composition of the adhesive layer (e.g., drug concentration, permeation enhancer, drug load, types of adhesives etc.).

It should be noted that the pharmaceutical composition formulated for the adhesive layer and/or the reservoir layer described herein is also a novel aspect of the present disclosure.

The transdermal delivery device herein can also be characterized by certain in vivo release profile, e.g., to provide a desired pharmacokinetic (PK) profile, e.g., any of those described herein. In some embodiments, the transdermal delivery device can be configured to provide a PK profile in a subject in need thereof, e.g., any of the PK profile described herein. In some embodiments, the transdermal delivery device is configured to provide a PK profile effective, for example, for treating a disease or disorder (e.g., described herein, such as PBA) in the subject.

The various aspects of transdermal delivery devices and formulations of the present disclosure can be combined in all possible combinations.

### Methods of Administering Dextromethorphan and Treatment

In various embodiments, the present disclosure also provides a method of using the transdermal delivery device or pharmaceutical compositions described herein, for example, for administering dextromethorphan to a subject in need thereof, e.g., those suffering from any of the diseases or disorders described herein.

Some embodiments are directed to a method of administering dextromethorphan to a subject (e.g., human subject) in need thereof. In some embodiments, the subject is sensitive to or otherwise intolerant to CYP2D6 inhibitors such as quinidine, e.g., having one or more side effects associated with quinidine, and/or is co-administered (or in need of) a drug whose metabolism is affected by CYP2D6 inhibitors such as quinidine. In some embodiments, the subject is sensitive to or otherwise intolerant to quinidine, e.g., with QTc prolongation. In some embodiments, the method comprises applying any of the transdermal delivery devices (e.g., those shown in [1]-[51] in the Brief Summary section) or pharmaceutical compositions to the subject, for example, to the skin of the subject. In some embodiments, the subject is not administered dextromethorphan through another source, for example, through oral administration. However, in some embodiments, the subject can also be supplemented with another source of dextromethorphan, for example, by co-administering an oral formulation of dextromethorphan to the subject. In some embodiments, the subject does not suffer from a cough and/or does not need an antitussive. In some embodiments, the subject is characterized as an extensive metabolizer. In some embodiments, the subject is characterized as a poor metabolizer. In some embodiments, the subject is not co-administered a CYP2D6 inhibitor. In some embodiments, the subject is not co-administered quinidine. In some embodiments, the subject is co-administered a CYP2D6 inhibitor such as quinidine, bupropion, etc.

Various dosing regimen are suitable for the methods herein. For example, in some embodiments, the method comprises administering a transdermal delivery device (e.g., described herein, such as those shown in [1]-[51] in the Brief Summary section) to the subject once a week (e.g., replaced every week) for a desired period of time. In some embodiments, the transdermal delivery device comprises about 150 mg to about 1000 mg of dextromethorphan. In some embodiments, the method can also comprise administering a transdermal delivery device (e.g., described herein) to the subject at a dosing frequency ranging from once a day to once a week, such as once in at least one day, for example, once a week, or 1, 2, 3, 4, 5, or 6 times a week for a desired period of time, preferably, the transdermal patch is applied to the subject once a week or twice a week. In some embodiments, the method can also comprise administering a transdermal delivery device (e.g., described herein, such as those shown in [1]-[51] in the Brief Summary section) to the subject once a week. For the avoidance of doubt, when it is said that the transdermal delivery device is applied to a subject once a week, it should mean that each application of the transdermal delivery device has a duration of about 1 week, i.e., continuously adheres to the skin of the subject for about 1 week, and that it can be replaced every week for the treatment period. Other expressions should be understood similarly.

The methods of administering dextromethorphan herein typically provide certain pharmacokinetic profile in a subject (e.g., human subject) in need thereof that is suitable (e.g., effective), for example, for treating a disease or disorder (e.g., any of those described herein such as PBA) in the subject, such as those described herein and in WO 2021/202329 A1.

The methods herein are not limited to a particular subject or a particular class of subjects. In some embodiments, the subject is characterized as an extensive metabolizer. In some embodiments, the subject is characterized as a poor metabolizer. In some embodiments, the subject is not co-administered a CYP2D6 inhibitor. In some embodiments, the subject is not co-administered quinidine. In some embodiments, the subject is co-administered a CYP2D6 inhibitor such as quinidine, bupropion, etc. However, in any of the embodiments described herein, the subject does not suffer from a cough and/or does not need an antitussive.

In some embodiments, the subject (e.g., human subject) is characterized as having a neurological disease or disorder. In some embodiments, the subject (e.g., human subject) is characterized as having one or more diseases or disorders selected from affective disorders, psychiatric disorders, cerebral function disorders, movement disorders, dementias, motor neuron diseases, neurodegenerative diseases, seizure disorders, and headaches. In some embodiments, the subject suffers from one or more diseases or disorders selected from depression, major depressive disorder, treatment resistant depression, treatment resistant bipolar depression, bipolar disorders including cyclothymia, seasonal affective disorder, mood disorders, chronic depression (dysthymia), psychotic depression, postpartum depression, premenstrual dysphoric disorder (PMDD), situational depression, atypical depression, mania, anxiety disorders, attention deficit disorder (ADD), attention deficit disorder with hyperactivity (ADDH), attention deficit/hyperactivity disorder (AD/HD), bipolar and manic conditions, obsessive-compulsive disorder, bulimia, obesity or weight-gain, narcolepsy, chronic fatigue syndrome, premenstrual syndrome, substance addiction or abuse, nicotine addiction, psycho-sexual dysfunction, pseudobulbar affect, and emotional lability. In some embodiments, the subject suffers from one or more diseases or disorders selected from Alzheimer's disease, prion-related diseases, cerebellar ataxia, spinocerebellar ataxia (SCA), spinal muscular atrophy (SMA), bulbar muscular atrophy, Friedrich's ataxia, Huntington's disease, Lewy body disease, Parkinson's disease, amyotrophic lateral sclerosis (ALS or Lou Gehrig's disease), multiple sclerosis (MS), multiple system atrophy, Shy-Drager syndrome, corticobasal degeneration, progressive supranuclear palsy, Wilson's disease, Menkes disease, adrenoleukodystrophy, cerebral autosomal dominant arteriopathy with subcortical infarcts and leukoencephalopathy (CADASIL), muscular dystrophies, Charcot-Marie-Tooth disease (CMT), familial spastic paraparesis, neurofibromatosis, olivopontine cerebellar atrophy or degeneration, striatonigral degeneration, Guillain-Barré syndrome, and spastic paraplesia. In any of the embodiments herein, the subject can suffer from pseudobulbar affect, depression (e.g., major depressive disorder or treatment resistant depression), stroke, traumatic brain injury, seizure, pain (e.g., post-operative pain, neuropathic pain), methotrexate neurotoxicity, Parkinson's disease, autism, or combinations thereof. In any of the embodiments herein, the subject can suffer from pseudobulbar affect.

### Methods of Treatment

Dextromethorphan are known to be useful for treating a variety of diseases or disorders. See e.g., Nguyen, L. et al., Pharmacology & Therapeutics 159:1022 (2016). Thus, in some embodiments, the present disclosure is also directed to a method of treating a disease or disorder in a subject in need thereof, for which administering dextromethorphan is beneficial. In some embodiments, the method comprises transdermally administering to the subject a therapeutically effective amount of dextromethorphan. In some embodiments, the administering comprises applying the transdermal delivery device (e.g., described herein, such as those shown in [1]-[51] in the Brief Summary section) to the skin of the subject. In some embodiments, the administering results in a PK profile described herein or any of those described in WO 2021/202329 A1. In some embodiments, the subject does not suffer from a cough and/or does not need an antitussive agent. In some embodiments, the subject is an extensive metabolizer of dextromethorphan. In some embodiments, the subject is a poor metabolizer of dextromethorphan. In some embodiments, the subject is sensitive or intolerant to CYP2D6 inhibitors. In some embodiments, the subject is sensitive to or otherwise intolerant to quinidine, e.g., with QTc prolongation. In some embodiments, the subject has one or more side effects associated with quinidine. In some embodiments, the subject is co-administered (or in need of) a drug whose metabolism is affected by a CYP2D6 inhibitor.

Various diseases and disorders are suitable to be treated by the methods herein. In some embodiments, the disease or disorder is a neurological disorder. Non-limiting exemplary neurological diseases or disorders include affective disorders, psychiatric disorders, cerebral function disorders, movement disorders, dementias, motor neuron diseases, neurodegenerative diseases, seizure disorders, and headaches.

Affective disorders that can be treated by methods herein include, but are not limited to, depression, major depressive disorder, treatment resistant depression and treatment resistant bipolar depression, bipolar disorders including cyclothymia, seasonal affective disorder, mood disorders, chronic depression (dysthymia), psychotic depression, postpartum depression, premenstrual dysphoric disorder (PMDD), situational depression, atypical depression, mania, anxiety disorders, attention deficit disorder (ADD), attention deficit disorder with hyperactivity (ADDH), and attention deficit/hyperactivity disorder (AD/HD), bipolar and manic conditions, obsessive-compulsive disorder, bulimia, obesity or weight-gain, narcolepsy, chronic fatigue syndrome, premenstrual syndrome, substance addiction or abuse, nicotine addiction, psycho-sexual dysfunction, pseudobulbar affect, and emotional lability.

Psychiatric disorders that can be treated by the methods herein include, but are not limited to, anxiety disorders, including but not limited to, phobias, generalized anxiety disorder, social anxiety disorder, panic disorder, agoraphobia, obsessive-compulsive disorder, and post-traumatic stress disorder (PTSD); mania, manic depressive illness, hypomania, unipolar depression, depression, stress disorders, somatoform disorders, personality disorders, psychosis, schizophrenia, delusional disorder, schizoaffective disorder, schizotypy, aggression, aggression in Alzheimer's disease, agitation, and agitation in Alzheimer's disease.

Substance addiction abuse that can be treated by the methods herein include, but is not limited to, drug dependence, addiction to cocaine, psychostimulants (e.g., crack, cocaine, speed, meth), nicotine, alcohol, opioids, anxiolytic and hypnotic drugs, cannabis (marijuana), amphetamines, hallucinogens, phencyclidine, volatile solvents, and volatile nitrites. Nicotine addiction includes nicotine addiction of all known forms, such as smoking cigarettes, cigars and/or pipes, and addiction to chewing tobacco.

Cerebral function disorders that can be treated by the methods herein include, but are not limited to, disorders involving intellectual deficits such as senile dementia, Alzheimer's type dementia, memory loss, amnesia/amnestic syndrome, epilepsy, disturbances of consciousness, coma, lowering of attention, speech disorders, voice spasms, Parkinson's disease, Lennox-Gastaut syndrome, autism, hyperkinetic syndrome, and schizophrenia. Cerebral function disorders also include disorders caused by cerebrovascular diseases including, but not limited to, stroke, cerebral infarction, cerebral bleeding, cerebral arteriosclerosis, cerebral venous thrombosis, head injuries, and the like where symptoms include disturbance of consciousness, senile dementia, coma, lowering of attention, and speech disorders.

Movement disorders that can be treated by the methods herein include, but are not limited to, akathisia, akinesia, associated movements, athetosis, ataxia, ballismus, hemiballismus, bradykinesia, cerebral palsy, chorea, Huntington's disease, rheumatic chorea, Sydenham's chorea, dyskinesia, tardive dyskinesia, dystonia, blepharospasm, spasmodic torticollis, dopamine-responsive dystonia, Parkinson's disease, restless legs syndrome (RLS), tremor, essential tremor, and Tourette's syndrome, and Wilson's disease.

Dementias that can be treated by the methods herein include, but are not limited to, Alzheimer's disease, Parkinson's disease, vascular dementia, dementia with Lewy bodies, mixed dementia, fronto-temporal dementia, Creutzfeldt-Jakob disease, normal pressure hydrocephalus, Huntington's disease, Wernicke-Korsakoff Syndrome, and Pick's disease.

Motor neuron diseases that can be treated by the methods herein include, but are not limited to, amyotrophic lateral sclerosis (ALS), progressive bulbar palsy, primary lateral sclerosis (PLS), progressive muscular atrophy, post-polio syndrome (PPS), spinal muscular atrophy (SMA), spinal motor atrophies, Tay-Sach's disease, Sandoff disease, and hereditary spastic paraplegia.

Neurodegenerative diseases that can be treated by the methods herein include, but are not limited to Alzheimer's disease, prion-related diseases, cerebellar ataxia, spinocerebellar ataxia (SCA), spinal muscular atrophy (SMA), bulbar muscular atrophy, Friedrich's ataxia, Huntington's disease, Lewy body disease, Parkinson's disease, amyotrophic lateral sclerosis (ALS or Lou Gehrig's disease), multiple sclerosis (MS), multiple system atrophy, Shy-Drager syndrome, corticobasal degeneration, progressive supranuclear palsy, Wilson's disease, Menkes disease, adrenoleukodystrophy, cerebral autosomal dominant arteriopathy with subcortical infarcts and leukoencephalopathy (CADASIL), muscular dystrophies, Charcot-Marie-Tooth disease (CMT), familial spastic paraparesis, neurofibromatosis, olivopontine cerebellar atrophy or degeneration, striatonigral degeneration, Guillain-Barré syndrome, and spastic paraplesia.

Seizure disorders that can be treated by the methods herein include, but are not limited to, epileptic seizures, nonepileptic seizures, epilepsy, febrile seizures; partial seizures including, but not limited to, simple partial seizures, Jacksonian seizures, complex partial seizures, and epilepsia partialis continua; generalized seizures including, but not limited to, generalized tonic-clonic seizures, absence seizures, atonic seizures, myoclonic seizures, juvenile myoclonic seizures, and infantile spasms; and status epilepticus.

Types of headaches that can be treated by the methods herein include, but are not limited to, migraine, tension, and cluster headaches.

Other neurological disorders that can be treated by the methods herein include, but are not limited to, Rett Syndrome, autism, tinnitus, disturbances of consciousness disorders, sexual dysfunction, intractable coughing, narcolepsy, cataplexy; voice disorders due to uncontrolled laryngeal muscle spasms, including, but not limited to, abductor spasmodic dysphonia, adductor spasmodic dysphonia, muscular tension dysphonia, and vocal tremor; diabetic neuropathy, chemotherapy-induced neurotoxicity, such as methotrexate neurotoxicity; incontinence including, but not limited, stress urinary incontinence, urge urinary incontinence, and fecal incontinence; and erectile dysfunction.

In some embodiments, the disease or disorder is pain, joint pain, pain associated with sickle cell disease, pseudobulbar affect, depression (including major depressive disorder, treatment resistant depression, etc.), disorders related to memory and cognition, schizophrenia, Parkinson's disease, amyotrophic lateral sclerosis (ALS), Rhett's syndrome, seizures, cough (including chronic cough), etc.

The methods herein can also be used to treat, or provide relief to, any type of pain including, but not limited to, musculoskeletal pain, neuropathic pain, cancer-related pain, acute pain, nociceptive pain, inflammatory pain, arthritis pain, complex regional pain syndrome, etc.

In some embodiments, the disease or disorder can be allodynia, treatment refractory hyperalgesia, dermatitis, pain, inflammation or inflammatory conditions, such as Crohn's disease, including pain associated with inflammation, psoriasis, cancer, viral infection, or as an adjuvant treatment for multiple myeloma.

In any of the embodiments described herein, the method can be for treating pseudobulbar affect, depression (e.g., major depressive disorder, treatment resistant depression, etc.), stroke, traumatic brain injury, seizure, pain (e.g., post-operative pain, neuropathic pain), methotrexate neurotoxicity, Parkinson's disease, autism, or a combination thereof.

Suitable dosing regimen, dosing amount, duration, transdermal delivery devices, etc. include any of those described herein in any combination. In any of the embodiments described herein, the subject can be a human subject.

In some specific embodiments, the present disclosure provides a method of treating pseudobulbar affect comprising applying the transdermal delivery device herein (e.g., those shown in [1]-[51] in the Brief Summary section) to a subject in need thereof. In some embodiments, the transdermal delivery device comprises about 150 mg to about 1000 mg of dextromethorphan. In some embodiments, the transdermal delivery device is applied once a week, e.g., for 1 week, 1 month, or any period of time desired. In some embodiments, the transdermal delivery device is applied 1, 2, 3, 4, 5, or 6 times in a week, e.g., for 1 week, 1 month, or any period of time desired. In some embodiments, the transdermal delivery device is applied to achieve any of the pharmacokinetic profile described herein. In some embodiments, the subject is not administered a CYP2D6 inhibitor. In some embodiments, the subject is not administered quinidine. In some embodiments, the subject does not suffer from a cough or need an antitussive effect. In some embodiments, the subject is characterized as a poor metabolizer. In some embodiments, the subject is characterized as an extensive metabolizer.

In some embodiments, the methods herein can further comprise administering to the subject an active agent other than dextromethorphan. For example, in some embodiments, the method described herein further comprises administering to the subject an antidepressant. In some embodiments, the antidepressant is selected from bupropion, hydroxybupropion, erythrohydroxybupropion, threohydroxybupropion, a metabolite or prodrug of any of these compounds, and combinations thereof. Other suitable antidepressants are described for example in U.S. Pat. No. 9,861,595, the content of which is incorporated by reference in its entirety. In some embodiments, the method described herein further comprises administering to the subject quinidine. In some embodiments, the method described herein further comprises administering to the subject a CYP2D6 inhibitor. In some embodiments, the method described herein further comprises administering to the subject one or more additional active agents selected from amlodipine, a capsaicinoid (e.g., capsaicin or an ester thereof), an opioid agonist (e.g., a μ-opiate analgesic (e.g., tramadol)), an adenosinergic agonist, 3-(3-dimethylamino-1-ethyl-2-methyl-propyl)-phenol, gabapentin, and pharmaceutically acceptable salts thereof. These additional agents can be administered simultaneously or sequentially. Further, these additional agents can be administered via the same or a different route. For example, in some embodiments, the additional agent can be administered transdermally or orally. However, in some embodiments, the additional agent can also be combined with dextromethorphan in the same transdermal delivery device.

Because the transdermal application described herein bypasses the first-pass liver metabolism, the methods herein can provide dextromethorphan to subjects who are on medications that might interfere with liver metabolism of dextromethorphan. In some embodiments, the method comprises administering to the subject desipramine, paroxetine, thioridazine, pimozide, digoxin, atazanavir, clarithromycin, indinavir, itraconazole, ketoconazole, and combinations thereof. However, in some embodiments, the subject is not administered any of desipramine, paroxetine, thioridazine, pimozide, digoxin, atazanavir, clarithromycin, indinavir, itraconazole, ketoconazole, and combinations thereof. In some embodiments, the method does not require determining whether the subject is an extensive metabolizer or poor metabolizer of dextromethorphan.

The present disclosure also provides the following non-limiting exemplary methods of transdermally administering dextromethorphan.

Typically, the methods herein are for treating a disease or disorder where administering dextromethorphan is beneficial. Suitable diseases or disorders that can be treated with the methods herein are described herein. In some embodiments, the methods herein are for treating a neurological disease or disorder in a subject in need thereof. Such neurological diseases or disorders include but not limited affective disorders, psychiatric disorders, cerebral function disorders, movement disorders, dementias, motor neuron diseases, neurodegenerative diseases, seizure disorders, and headaches. In some embodiments, the methods are for treating pseudobulbar affect, depression (e.g., major depressive disorder or treatment resistant depression), stroke, traumatic brain injury, seizure, pain, methotrexate neurotoxicity, Parkinson's disease, autism, or a combination thereof. In some embodiments, the subject does not suffer from a cough and/or does not need an antitussive.

Nuedexta® tablets were approved by the FDA for treating pseudobulbar affect or PBA, see the Prescribing Information of Nuedexta®, June 2019 version, the content of which is herein incorporated by reference in its entirety. As stated in the Nuedexta Prescribing Information, PBA occurs secondary to a variety of otherwise unrelated neurologic conditions, and is characterized by involuntary, sudden, and frequent episodes of laughing and/or crying. PBA episodes typically occur out of proportion or incongruent to the underlying emotional state. PBA is a specific condition, distinct from other types of emotional lability that may occur in patients with neurological disease or injury.

In some specific embodiments, the methods herein are for treating PBA in a subject in need thereof. In some embodiments, the subject also suffers from a neurodegenerative disease such as amyotrophic lateral sclerosis, multiple sclerosis, Parkinson's disease, and/or Alzheimer's disease, stroke, or a brain injury, such as traumatic brain injury.

The methods herein typically comprise transdermally delivering to the subject in need thereof a therapeutically effective amount of dextromethorphan. In some embodiments, the method comprises transdermally delivering to the subject in need thereof a daily dose of about 15 mg to about 50 mg (e.g., about 15 mg, about 20 mg, about 30 mg, about 40 mg, about 50 mg, or any values or ranges between the recited values, such as about 20-50 mg, about 30-50 mg, or about 20-40 mg, etc.) of dextromethorphan. In some embodiments, the daily dose is about 20 mg to 40 mg of dextromethorphan, such as about 35 mg. In some embodiments, the daily dose can also be higher than 50 mg, such as about 60 mg, up to about 100 mg of dextromethorphan. In some embodiments, the daily dose can be lower than 15 mg, for example, about 5 mg, about 10 mg, or about 5-10 mg of dextromethorphan. The daily dose of dextromethorphan is typically delivered by applying a transdermal delivery device or patch or adhesive composition/formulation herein, e.g., any of those described herein (e.g., those shown in [1]-[51] in the Brief Summary section), to the subject. As discussed herein, the transdermal patches herein are advantageous in that each application can provide a desired daily dose of dextromethorphan for seven days or more. Thus, the methods herein can provide better patient compliance and convenience, with the advantageous once a week dosing regimen.

In some embodiments, the methods herein can also be characterized by a unique in vivo pharmacokinetic (PK) profile described herein. As shown in more details in the Examples section, applying an exemplary patch to human subjects once a week provided a therapeutically effective plasma concentrations for seven days or more, similar to the reference patch applied once a day, see also WO 2021/202329 A1. Treatment of the diseases or disorders herein with the novel PK profiles described herein is by itself a novel feature of the present disclosure. These unique PK profiles provide many advantages, which include but not limited to a more accurate dosing, less frequent dosing, reduced potential for side effects associated with quinidine and/or higher exposure (e.g., Cₘₐₓ) of dextromethorphan, reduced pill burden, and better patient compliance. In some embodiments, the PK profile is characterized as having one or more of the following characteristics: (1) an AUC_{0-24, DXM} at day-7 or steady state stage between about 180 h*ng/mL to about 2000 h*ng/mL, for example, about 200 h*ng/ml to about 600 h*ng/ml or about 300 h*ng/ml to about 500 h*ng/ml; (2) a C_{Avg, DXM} at day-7 or steady state stage between about 8 ng/mL to about 100 ng/mL, e.g., about 10 ng/mL to about 20 ng/mL, such as about 15 ng/ml; (3) a C_{min, DXM} at day-7 or steady state stage between about 6 ng/mL to about 65 ng/ml, e.g., about 6 ng/ml to about 20 ng/ml; and (4) a C_{max, DXM} at day-7 or steady state stage between about 8 ng/ml to about 90 ng/ml, e.g., about 10 ng/ml to about 30 ng/mL. In some embodiments, the PK profile is characterized as having one or more of the following characteristics: a) an AUC_{0-24, DXM} at day-7 or steady state stage between about 200 h*ng/ml to about 600 h*ng/ml; b) a C_{Avg, DXM} at day-7 or steady state stage about 10 ng/ml to about 20 ng/ml, such as about 15 ng/ml; c) a C_{min, DXM} at day-7 or steady state stage between about 6 ng/mL to about 20 ng/ml; and/or d) a C_{max, DXM} at day-7 or steady state stage between about 10 ng/mL to about 30 ng/mL.

In some embodiments, the methods herein can be particularly useful and advantageous for treating certain subjects. Patients having neurological disorder(s) often have multiple comorbidities and/or are being treated with numerous other medications. For example, the clinical trials (controlled or uncontrolled) conducted for PBA were based on patient population having also Amyotrophic lateral sclerosis (ALS), Multiple sclerosis (MS) and a variety of other underlying neurological conditions including stroke and traumatic brain injury. Thus, patients having PBA are typically also treated with other medications such as medications for treating ALS, MS, stroke, and traumatic brain injury, etc. The use of Nuedexta® tablets, or similar strategies of using a CYP2D6 inhibitor to enhance dextromethorphan plasma concentration, is limited and may cause various restrictions and drug-drug interactions for such patients. Some of the side effects or drugs that are affected by a CYP2D6 inhibitor are described in the Prescribing Information of Nuedexta®, June 2019 version, the content of which is herein incorporated by reference in its entirety. For example, the Prescribing Information of Nuedexta® describes the following contraindications: 1) Patients with a history of quinidine, quinine or mefloquine-induced thrombocytopenia, hepatitis, or other hypersensitivity reactions such as bone marrow depression or lupus-like syndrome; 2) Patients with known hypersensitivity to dextromethorphan; 3) Use with an MAOI or within 14 days of stopping an MAOI. Allow 14 days after stopping NUEDEXTA before starting an MAOI; 4) Prolonged QT interval, congenital long QT syndrome, history suggestive of torsades de pointes, or heart failure; 5) Complete atrioventricular (AV) block without implanted pacemaker, or patients at high risk of complete AV block; and 6) Concomitant use with drugs that both prolong QT interval and are metabolized by CYP2D6 (e.g., thioridazine or pimozide). The Prescribing Information of Nuedexta® also describes various warnings and precautions including a) thrombocytopenia or other hypersensitivity reactions; b) Hepatitis; c) QT prolongation; d) Left ventricular hypertrophy (LVH) or left ventricular dysfunction (LVD); e) CYP2D6 substrate; f) dizziness; g) serotonin syndrome; and h) anticholinergic effects of quinidine. Many of these contraindications, warnings and precautions are associated with quinidine. For example, the Prescribing Information of Nuedexta® describes that “Quinidine can cause immune-mediated thrombocytopenia that can be severe or fatal”; “Quinidine has also been associated with a lupus-like syndrome involving polyarthritis”; “Other associations include rash, bronchospasm, lymphadenopathy, hemolytic anemia, vasculitis, uveitis, angioedema, agranulocytosis, the sicca syndrome, myalgia, elevation in serum levels of skeletal-muscle enzymes, and pneumonitis”; “Hepatitis, including granulomatous hepatitis, has been reported in patients receiving quinidine;” quinidine can also cause “accumulation of parent drug and/or failure of active metabolite formation may decrease the safety and/or the efficacy of drugs used concomitantly with NUEDEXTA that are metabolized by CYP2D6”; “potentially fatal cardiac arrhythmia, including torsades de pointes, can occur at quinidine exposures that are possible from NUEDEXTA overdose”. Chronic quinidine toxicity may be possible with NUEDEXTA treatment. Further, a variety of drug s can have an effect on the pharmacological effect of quinidine, such as CYP3A4 Inhibitor, P-glycoprotein blocker, drugs have direct effects on QTc or are arrhythmogenic themselves, low serum potassium or moderately low potassium levels in association with diuretics, which can restrict the use of NUEDEXTA. Because quinidine inhibits CYP2D6, a variety of drug-drug interaction is also possible for CYP2D6 substrates such as desipramine, paroxetine. As described in the Prescribing Information of Nuedexta®, “in cases of prodrugs whose actions are mediated by the CYP2D6-produced metabolites (for example, codeine and hydrocodone, whose analgesic and antitussive effects appear to be mediated by morphine and hydromorphone, respectively), it may not be possible to achieve the desired clinical benefits in the presence of NUEDEXTA due to quinidine-mediated inhibition of CYP2D6.” Quinidine is also an inhibitor of p-glycoprotein, which can significantly affect the plasma level of drugs that are p-glycoprotein substrates, such as digoxine. In sum, because of the various potential side effects associated with quinidine, there exists an unmet medical need, at least with respect to the treatment of PBA in patient populations that have one or more restrictions and/or side effects associated with quinidine or in general CYP2D6 inhibitors.

The transdermal delivery route described herein does not require the use of quinidine or other CYP2D6 inhibitors and thus can be advantageously used for treating patients without the restrictions associated with quinidine or CYP2D6 inhibitors. For example, in some embodiments, the methods herein can treat subjects that are sensitive or intolerant to quinidine or in general to CYP2D6 inhibitors. In some embodiments, the subject can be sensitive or intolerant to CYP2D6 inhibitors. In some embodiments, the subject can be sensitive or intolerant to quinidine. In some embodiments, the subject has one or more side effects associated with quinidine. In some embodiments, the subject is co-administered a drug whose metabolism is affected by a CYP2D6 inhibitor. In some embodiments, the subject is co-administered a drug whose metabolism is affected by quinidine. In some embodiments, the subject is co-administered a drug that can affect the pharmacological effect of quinidine, such as a CYP3A4 inhibitor (e.g., atazanavir, clarithromycin, indinavir, itraconazole, ketoconazole, nefazodone, nelfinavir, ritonavir, saquinavir, telithromycin, amprenavir, aprepitant, diltiazem, erythromycin, fluconazole, fosamprenavir, grapefruit juice, and verapamil). In some embodiments, the subject can be further treated with a Selective Serotonin Reuptake Inhibitor (such as fluoxetine), a tricyclic antidepressant (such as clomipramine and imipramine), and/or a monoamine oxidase inhibitor (MAOI).

Further, as the transdermal delivery route described herein does not require the use of quinidine or other CYP2D6 inhibitors, the transdermal delivery devices or formulations herein can be conveniently administered to transdermally deliver dextromethorphan to a subject with or without first determining whether the subject is a poor metabolizer, an intermediate metabolizer, or an extensive metabolizer of dextromethorphan. In a poor metabolizer, the addition of quinidine or other CYP2D6 inhibitors is not expected to have a significant effect on the plasma exposure of dextromethorphan, but such addition would nonetheless expose the subject to the potential side effects associated with quinidine or other CYP2D6 inhibitors. The transdermal delivery methods described herein do not suffer from such drawbacks. In some embodiments, the methods herein can treat a subject without first determining whether the subject is a poor metabolizer, an intermediate metabolizer, or an extensive metabolizer of dextromethorphan. In some embodiments, the method herein can treat a subject that is an extensive metabolizer. In some embodiments, the method herein can treat a subject that is a poor metabolizer. In some embodiments, the method herein can also comprise determining whether a subject is a poor metabolizer, an intermediate metabolizer, or an extensive metabolizer of dextromethorphan, and administering to the subject an appropriate daily dose of dextromethorphan to the subject. For example, in some embodiments, the daily dose can be adjusted such that the transdermal delivery results in a therapeutically effective plasma concentration of dextromethorphan in the subject. In some embodiments, the daily dose can be adjusted such that the transdermal delivery results in any of the PK profile described herein. For example, in some embodiments, the present disclosure provides a method of treating a neurological disease or disorder (e.g., any of those described herein) in a subject in need thereof, the method comprising (a) applying a first transdermal patch (e.g., those shown in [1]-[51] in the Brief Summary Section) to the subject at a dosing frequency of once a day to once a week to deliver a first daily dose (typically about 15 mg to about 50 mg) of dextromethorphan to the subject; (b) determining whether the applying results in any of the pharmacokinetic profile disclosed herein; and optionally (c) adjusting the first daily dose upper or lower such that the applying results in one or more of the pharmacokinetic profile disclosed herein. Suitable transdermal patches and dosing regimens include any of those described herein.

As discussed herein, it is expected that the methods herein would at least produce a reduced incidence of side effects associated with high exposure (e.g., Cₘₐₓ, AUC, etc.) of dextromethorphan. Thus, in some embodiments, the methods herein can also be advantageously used to treat a subject who has one or more side effects associated with high exposure (e.g., Cₘₐₓ, AUC, etc.) of dextromethorphan.

The methods herein can be used in combination with other medications. For example, in some embodiments, the method can further comprise administering to the subject an antidepressant. In some embodiments, the antidepressant is bupropion, hydroxybupropion, erythrohydroxybupropion, threohydroxybupropion, a metabolite or prodrug of any of these compounds, and combinations thereof. In some embodiments, the method can further comprise administering to the subject one or more additional active agents selected from amlodipine, a capsaicinoid (e.g., capsaicin or an ester thereof), an opioid agonist (e.g., a μ-opiate analgesic (e.g., tramadol)), an adenosinergic agonist, 3-(3-dimethylamino-1-ethyl-2-methyl-propyl)-phenol, gabapentin, and pharmaceutically acceptable salts thereof. Typically, the methods herein do not administer to the subject quinidine. However, in some embodiments, quinidine can also be administered. These additional agents can be administered simultaneously or sequentially. Further, these additional agents can be administered via the same or a different route. For example, in some embodiments, the additional agent can be administered transdermally or orally. However, in some embodiments, the additional agent can also be combined with dextromethorphan in the same transdermal delivery device.

### DEFINITIONS

As used herein, the term “about” modifying an amount related to the invention refers to variation in the numerical quantity that can occur, for example, through routine testing and handling; through inadvertent error in such testing and handling; through differences in the manufacture, source, or purity of ingredients/materials employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

As used herein, the term “cumulative drug permeated” refers to the total amount of drug permeated per square centimeter during a given period of time. Unless otherwise obvious from context, “cumulative drug permeated” at a given time (e.g., at 24 hours post administration) refers to the total amount of drug permeated per square centimeter from time 0 (i.e., time of administration) to the given time. Unless otherwise obvious from context, “cumulative drug permeated” refers to the arithmetic mean value measured and/or calculated in accordance with the methods described herein. The term “mean value” as used herein, when not specified, also refers to arithmetic mean value, unless contradictory to common practice in the field.

As used herein, the term “flux” refers to the quantity of the drug permeated skin per unit area per unit time. Unless otherwise obvious from context, “flux” refers to the arithmetic mean value measured and/or calculated in accordance with the methods described herein. A typical unit of flux is milligram per square centimeter per hour or per day. Dextromethorphan flux per day as used herein should be understood as the arithmetic mean value of the cumulative dextromethorphan permeated at 24 hours post application, measured and/or calculated in accordance with the methods described herein.

Flux rate as referenced in this patent application can mean that measured by either in vivo or in vitro methods. One way to measure flux is to place the transdermal delivery device or formulation on a known skin area of a human volunteer and measure how much drug can permeate across skin within certain time constraints. Those skilled in the art would understand that in some cases, the absolute value of in vitro flux can be several fold different when measured using a different cadaver source. As used herein, when specifically referenced as measured by in vitro method using human cadaver skin, the flux rate should be understood as measured in accordance with the method described in Example 4. For example, a patch tested in Example 4 can be used as a reference patch, which when tested in a method in accordance with Example 4, should yield the same flux as observed in Example 4, within experimental error generally accepted by those skilled in the art. Although an in vitro method uses human epidermal membrane obtained from a cadaver, rather than measure drug flux across the skin using human volunteers, it is generally accepted by those skilled in the art that results from a properly designed and executed in vitro test can be used to estimate or predict the results of an in vivo test with reasonable reliability.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

The term “therapeutically effective amount,” as used herein, refers to that amount of a therapeutic agent (e.g., dextromethorphan) sufficient to result in amelioration of one or more symptoms of a disorder or condition (e.g., PBA), or prevent appearance or advancement of a disorder or condition, or cause regression of or cure from the disorder or condition.

The term “subject” (alternatively referred to herein as “patient”) as used herein, refers to an animal, preferably a mammal, most preferably a human, who has been the object of treatment, observation or experiment.

As used herein, applying or administering the transdermal delivery device herein should be understood as in accordance with how such transdermal delivery device is normally applied or administered, e.g., to the skin of a human subject.

### EXAMPLES

### Example 1. Preparation of Dextromethorphan Transdermal Patch

This example shows one procedure for preparing a single layer dextromethorphan drug-in-adhesive patch. Dextromethorphan base (“DXM”) is generally commercially available. Alternatively, dextromethorphan base can be prepared by conversion of dextromethorphan hydrobromide into the free base, for example, using a 1:1 molar ratio of NaOH.

Preparation of Formulation A: Formulation A has the following ingredients (weight percentages are based on dry weight)

| Ingredient | % w/w |
|---|---|
| Dextromethorphan | 30 |
| Acrylic adhesive (DuroTak ® 387- | 50 |
| 2287) | |
| Plasdone K29/32 | 10 |
| Isopropyl myristate | 10 |

Patches based on Formulation A were prepared according to the general procedure below:
In a disposable beaker-1, add in isopropyl alcohol (IPA) and add in Plasdone K29/32, stir for 30 min or until dissolve. Add in weighed IPM (isopropyl myristate). Mix till dissolve.
In disposable beaker-2, weigh and add in ethyl acetate. Add in DXM and mix till the dispersion is well-mixed. Then weigh and add in DuroTak® 387-2287.
Add content of beaker-1 to beaker-2 while mixing. Mix them till homogeneous.
Cast on backing film (3M beige film 9723NR):
Drying—drying of cast adhesive film in oven at 85 °C for 10 min.
Lamination—laminate release liner onto the dried adhesive film.
Die cutting (to 70 cm² size) and Pouching.

### Example 2. Preparation of Dextromethorphan Transdermal Patch Containing a Reservoir Layer

This example shows one procedure for preparing dextromethorphan drug-in-adhesive patch having a skin contacting drug-in-adhesive layer and a reservoir layer.

The ingredients for the skin contacting drug-in-adhesive layer and reservoir layer are shown below, weight percentages are based on dry weight:

% w/w

| | Skin Contacting | |
|---|---|---|
| | drug-in-adhesive | Reservoir layer (2^{nd} |
| Ingredient | layer (1^{st} layer) | Layer) |
| Dextromethorphan | 8 | 30 |
| Acrylic adhesive | 72 | 41 |
| DuroTak ® 387- | | |
| 2287) | | |
| Plasdone K29/32 | 10 | 19 |
| Isopropyl myristate | 10 | 10 |

Patches in this Example were prepared according to the general procedure below:

### Reservoir Layer:

In a disposable beaker-1, add in IPA and add in Plasdone K29/32, stir for 30 min or until dissolve. Add in weighed IPM (isopropyl myristate). Mix till dissolve.
In a disposable beaker-2, weigh and add in ethyl acetate. Add in DXM and mix till the dispersion is well-mixed. Then weigh and add in DuroTak® 387-2287.
Add content of beaker-1 to beaker-2 while mixing. Mix them till homogeneous.
Cast on backing film (3M beige film 9723NR)·
Drying—drying of cast adhesive film in oven at 85 °C for 10 min.

### Skin-Contacting Drug-In-Adhesive Layer:

In a disposable beaker-1, add in IPA and add in Plasdone K29/32, stir for 30 min or until dissolve. Add in weighed IPM (isopropyl myristate). Mix till dissolve.
In a disposable beaker-2, weigh and add in ethyl acetate. Add in DXM and mix till the DXM gets dissolved. Then weigh and add in DuroTak® 387-2287.
Add content of beaker-1 to beaker-2 while mixing. Mix them till homongeneous.
Cast on backing film (3M beige film 9723NR)·
Drying—drying of cast adhesive film in oven at 85 °C for 10 min.

### Bi-layer Patch Construction:

Laminate the skin-contacting drug-in-adhesive layer onto the reservoir layer films. The thickness of the skin-contacting drug-in-adhesive layer is about 3 mils, and the thickness of the reservoir layer is about 4 mils. For each 100 mg, the reservoir layer is about 57.14 mg and the skin-contacting drug-in-adhesive layer is about 42.86 mg.
Laminate release liner onto the dried adhesive bi-layer film of above.
Die cutting (to 70 cm² size) and Pouching.

### Example 3. Preparation of a Reference Dextromethorphan Transdermal Patch

A single layer drug-in-adhesive patch was prepared according to procedures described in WO2021/202329, published Oct. 7, 2021, as a reference patch. Formulation E1 contains, by dry weight percentage, about 10% of dextromethorphan base, about 10% of isopropyl myristate, about 70% of polyacrylate adhesive (DuroTak® 387-2287), and about 10% of crystallization inhibitor Plasdone K-29/32 (a polyvinylpyrrolidone). The ingredients were blended with isopropanol to form a homogenous solution. This wet formulation has the following ingredients, about 63.1% of polyacrylate adhesive (DuroTak® 387-2287, has about 50% solid content), about 4.5% Plasdone K-29/32 (a polyvinylpyrrolidone), about 4.5% isopropyl myristate, about 4.5% dextromethorphan base, and about 23.4% isopropyl alcohol. This wet formulation was casted onto a release liner (3 Mil PET 8310, silicone coated polyester film) and then dried. The dried casting was then laminated to a patch backing film, Scotchpak 9733 PET film. The patch was die-cut into desired size. In one example, this formulation was used to prepare transdermal patches, for example, with about 56 mg of dextromethorphan base and a size of about 70 cm².

### Example 4. Transdermal Flux Test

Transdermal flux of Dextromethorphan from the patch was tested using human cadaver skin by Franz Diffusion Cell method. See e.g., WO2021/202329.

Patch prepared in Examples 1 and 2 were used for a skin permeation study using the following protocol:
Franz cell assembly—Logan Instruments (6-cell unit)
Each cell has 12 mL volume, 1.5 cm diameter orifice
Receptor medium is a phosphate buffer solution (PBS) pH 7.4
Cell temperature is maintained at 37 °C
Sampling method: take 1.5 mL for HPLC assay, empty cell, replace with fresh medium
Sampling time points: 4, 8, 12, 24, 48, 72, 96, 120, 144, 168 and 192 hours
Assay method for media: HPLC based.

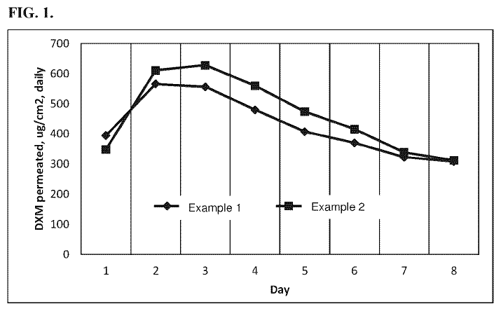

RESULTS of the study are presented in Table 1 below (See also FIG. 1). The skin permeation experiments were conducted up to 8 days. The values presented are cumulative amount of DXM permeated per cm² (i.e., μg/cm²).

TABLE 1

| Day | Example 1 | Example 2 |
|---|---|---|
| 1 | 395.2 | 349.0 |
| 2 | 566.1 | 611.3 |
| 3 | 556.9 | 628.1 |
| 4 | 480.0 | 560.4 |
| 5 | 407.9 | 474.3 |
| 6 | 370.3 | 416.4 |
| 7 | 323.1 | 339.0 |
| 8 | 308.7 | 312.4 |

### Example 5. Stability Study

The patches prepared in Examples 1 and 2 were also tested for storage stability under two conditions: 25 °C/60% RH (relative humidity) and 40 °C/75% RH. The results are shown in the tables 2 and 3 below.

TABLE 2
Stability of Patches from Example 1

| | |
|---|---|
| | Example 1: Dextromethorphan Transdermal |
| | Patch, 70 cm² |
| Product Name | 7-Day Patch (Single Layer) |

Batch No. 140-210423; Batch Size: 250 grams

| | | | |
|---|---|---|---|
| Stability storage | Initial | 25 °C/60% RH | 40 °C/75% RH |
| conditions | | | |
| Test Parameter/Month | 0 month | 1 month | 1 month |
| Peel Adhesion, g/in | 930 | 920 | 980 |
| Cold Flow | Conforms | Conforms | Conforms |
| Release Liner Removal | 17 | 18 | 16 |
| Force, g/in | | | |
| Assay of | 100.0 | 100.0 | 99.5 |
| Dextromethorphan, % | | | |
| claim | | | |
| Impurities of | Not | Not | 0.11% |
| Dextromethorphan, % | Detectable | Detectable | |

As shown in Table 2, the single layer patches prepared according to Example 1 were found to have good storage stability, and good peel adhesion and release liner removal force. In addition, the dextromethorphan content remained unchanged for at least 1 month under two storage conditions.

TABLE 3
Stability of Patches from Example 2

| | |
|---|---|
| | Example 2: Dexamethasone Transdermal |
| | Patch, 70 cm² |
| Product Name | 7-Day Patch (Bi-Layer) |

Batch No. 140-210501; Batch Size: 250 grams

| | | | |
|---|---|---|---|
| Stability storage conditions | Initial | 25 °C/60% RH | 40 °C/75% RH |
| Test Parameter/Month | 0 month | 1 month | 1 month |
| Peel Adhesion, g/in | 1030 | 1005 | 1010 |
| Cold Flow | Conforms | Conforms | Conforms |
| Release Liner Removal | 19 | 19 | 18 |
| Force, g/in | | | |
| Assay of | 100.0 | 100.0 | 99.6 |
| Dexamethasone, % | | | |
| claim | | | |
| Impurities of | Not | Not | 0.12% |
| Dexamethasone, % | Detectable | Detectable | |

As shown in Table 3, the bilayer patches prepared according to Example 2 were also found to have good storage stability, and good peel adhesion and release liner removal force. In addition, the dextromethorphan content remained unchanged for at least 1 month under two storage conditions.

### Example 6. In Vivo Pharmacokinetic Studies

This example concerns an Open-Label, Randomized, Three-Treatment, Three-Period, Three-Sequence, Crossover, Comparative Bioavailability Study of Single Dose of Test Products (T₁ & T₂) Dextromethorphan Extended-Release Transdermal Patch (Once a week) 35 mg/24 hr delivery with Multiple Dose of Reference (R) Dextromethorphan Transdermal Patch (once a day) 35 mg/24 hr delivery in Healthy Adult Human Male Subjects under Fasting Condition. T₁ refers to the single-layer patch according to Example 1 (70 cm², 35 mg/day for once a week dosing). T₂ refers to the bi-layer patch according to Example 2 (70 cm², 35 mg/day for once a week dosing). The Reference patch refers to the patch prepared according to Example 3 (70 cm², 35 mg/day for once a day dosing). The number of subjects in this study are 12.

The plasma concentrations of dextromethorphan and its active metabolite dextrorphan were measured by a fully validated analytical procedure. Statistical analysis was performed to evaluate the bioavailability of the test formulation relative to that of the reference product for dextromethorphan and dextrorphan only. For details of analytical procedures and statistical analysis, see WO2021/202329.

Tables 4A-4C show the results from this study.

TABLE 4A
Comparison of Dextromethorphan PK Profile in Subjects Treated
with Patches of Example 1 (T1) Vs Reference Patches

| | Geometric | | Geometric | | Geometric | | Intra | |
|---|---|---|---|---|---|---|---|---|
| Pharmacokinetic | LSM Test | | LSM | | LSM | | Subject | |
| Parameter | (T1) | N_{T} | Reference (R) | N_{R} | Ratio (%) | 90% CI | CV % | Power |
| Cₘₐₓ (ng/mL) | 18.39 | 9 | 21.04 | 9 | 87.38 | 56.16-135.94 | 48.96 | 18.66 |
| AUC₍₀₋ₜ₎ (h*ng/mL) | 1995.04 | 9 | 1836.99 | 9 | 108.60 | 84.91-138.91 | 26.25 | 43.10 |
| AUC_{(0-inf)} (h*ng/mL) | 2011.16 | 9 | 1894.32 | 9 | 106.17 | 84.40-133.56 | 24.42 | 47.94 |

N_{T}, N_{R}: Number of Subjects used for 90% CI calculation for Test & Reference respectively
LSM: Least Square Mean;
CI: Confidence Interval;
CV: Coefficient of Variation

TABLE 4B
Comparison of Dextromethorphan PK Profile in Subjects Treated
with Patches of Example 2 (T2) Vs Reference Patches

| | Geometric | | Geometric | | Geometric | | Intra | |
|---|---|---|---|---|---|---|---|---|
| Pharmacokinetic | LSM Test | | LSM | | LSM | | Subject | |
| Parameter | (T2) | N_{T} | Reference ® | N_{R} | Ratio (%) | 90% CI | CV % | Power |
| Cₘₐₓ (ng/mL) | 19.27 | 9 | 21.04 | 9 | 91.59 | 42.91-195.50 | 93.92 | 10.97 |
| AUC₍₀₋ₜ₎ (h*ng/mL) | 1840.23 | 9 | 1836.99 | 9 | 100.18 | 64.20-156.32 | 49.33 | 18.50 |
| AUC_{(0-inf)} (h*ng/mL) | 1950.12 | 9 | 1894.32 | 9 | 102.95 | 70.22-150.92 | 41.80 | 22.45 |

N_{T}, N_{R}: Number of Subjects used for 90% CI calculation for Test & Reference respectively
LSM: Least Square Mean;
CI: Confidence Interval;
CV: Coefficient of Variation

TABLE 4C
Comparison of Dextromethorphan PK Profile in Subjects Treated
with Patches of Example 1 (T1) Vs Example 2 (T2)

| | Geometric | | Geometric | | Geometric | | Intra | |
|---|---|---|---|---|---|---|---|---|
| Pharmacokinetic | LSM Test | | LSM Test | | LSM Ratio | | Subject | |
| Parameter | (T1) | N_{T1} | (T2) | N_{T2} | (%) | 90% CI | CV % | Power |
| Cₘₐₓ (ng/mL) | 21.14 | 12 | 20.61 | 12 | 102.57 | 71.81-146.49 | 50.47 | 25.51 |
| AUC₍₀₋ₜ₎ (h*ng/mL) | 2405.20 | 12 | 2272.77 | 12 | 105.83 | 83.69-133.82 | 32.15 | 46.50 |
| AUC_{(0-inf)} (h*ng/mL) | 2505.99 | 12 | 2452.05 | 12 | 102.20 | 80.38-129.94 | 32.93 | 44.98 |

N_{T1}, N_{T2}: Number of Subjects used for 90% CI calculation for Test (T₁) & Test (T₂) respectively
LSM: Least Square Mean;
CI: Confidence Interval;
CV: Coefficient of Variation

In addition to the pharmacokinetic studies, safety observations were also made. All of the subjects were found to be in normal health status at the time of post-study medical examination. No serious/significant adverse events were reported during the entire duration of the study.

Based on the study results, it was concluded that the key pharmacokinetic parameters of Test Products (T1 & T2) [Dextromethorphan Extended-Release Transdermal Patch (Once a week) 35 mg/24 hr delivery] and Multiple Dose of Reference Product (R) [Dextromethorphan Transdermal Patch (once a day) 35 mg/24 hr delivery] were comparable for dextromethorphan.

It was unexpected that these test products, which has drug particles in the adhesive layers, showed similar results to the reference product. This shows for the first time that it is possible to have a once a week dosing regimen that can transdermally deliver a therapeutically effective amount of dextromethorphan to a subject, which would be convenient for patients as these patches only need to be replaced once a week rather than daily.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A transdermal patch comprises:
i. a backing layer; and
ii. a drug-in-adhesive layer comprising 1) dextromethorphan in an amount of at least 15%, preferably, about 15% to about 50% by weight; 2) isopropyl myristate in an amount of about 6% to about 12% by weight; 3) a pressure sensitive adhesive, preferably, an acrylate based pressure sensitive adhesive, in an amount of about 20% to about 60% by weight; and 4) a solubilizer in an amount of about 6% to about 25% by weight, wherein at least a portion of the dextromethorphan is in a solid state dispersed in the pressure sensitive adhesive, preferably,
the transdermal patch has an active surface area of about 30 cm² to about 150 cm².

2-19. (canceled)

20. A transdermal patch comprising:
i. a backing layer;
ii. a skin-contacting drug-in-adhesive layer comprising 1) dextromethorphan in an amount of about 6% to about 12% by weight; 2) isopropyl myristate in an amount of about 6% to about 12% by weight; 3) a first pressure sensitive adhesive, preferably, an acrylate based pressure sensitive adhesive, in an amount of about 65% to about 85% by weight; and 4) a crystallization inhibitor in an amount of about 6% to about 12% by weight, and
iii. a reservoir layer comprising 1) dextromethorphan in an amount of at least 15% by weight; 2) isopropyl myristate in an amount of about 6% to about 12% by weight; (3) a second pressure sensitive adhesive in an amount of about 20% to about 60% by weight; and (4) a solubilizer in an amount of about 6% to about 25% by weight,
wherein at least a portion of the dextromethorphan in the reservoir layer is in a solid state dispersed in the second pressure sensitive adhesive, and
the first and second pressure sensitive adhesive are the same or different, preferably the same,
preferably, the transdermal patch has an active surface area of about 30 cm² to about 150 cm².

21-51. (canceled)

52. A method of treating a neurological disease or disorder in a subject in need thereof, the method comprising applying the transdermal patch of claim 1 to the subject.

53. The method of claim 52, comprising applying the transdermal patch to transdermally deliver a therapeutically effective amount of dextromethorphan to the subject.

54. The method of claim 52, comprising applying the transdermal patch to transdermally deliver a daily dose of about 15 mg to about 50 mg (e.g., about 15 mg, about 20 mg, about 30 mg, about 40 mg, about 50 mg, or any values or ranges between the recited values, such as about 20-50 mg, about 30-50 mg, or about 20-40 mg, etc.) of dextromethorphan to the subject.

55. The method of claim 54, wherein the daily dose is about 35 mg of dextromethorphan.

56. The method of claim 52, wherein the transdermal patch is applied to the subject at a dosing frequency ranging from once a day to once a week, preferably, the transdermal patch is applied to the subject once a week or twice a week.

57. The method of claim 52, wherein the neurological disease or disorder is pseudobulbar affect, depression, such as major depressive disorder or treatment resistant depression, stroke, traumatic brain injury, seizure, pain, methotrexate neurotoxicity, Parkinson's disease, autism, or a combination thereof.

58. The method of claim 57, wherein the neurological disease or disorder is pseudobulbar affect.

59. The method of claim 52, wherein the subject does not suffer from a cough and/or does not need an antitussive.

60. The method of claim 52, wherein the subject is characterized as an extensive metabolizer of dextromethorphan.

61. The method of claim 52 wherein the subject is characterized as a poor metabolizer of dextromethorphan.

62. The method of claim 52, wherein the subject is sensitive or intolerant to CYP2D6 inhibitors.

63. The method of claim 52, wherein the subject has one or more side effects associated with quinidine.

64. The method of claim 52, wherein the subject is co-administer red a drug whose metabolism is affected by a CYP2D6 inhibitor.

65. The method of claim 52, further comprising administering to the subject an antidepressant.

66. The method of claim 65, wherein the antidepressant is selected from bupropion, hydroxybupropion, erythrohydroxybupropion, threohydroxybupropion, a metabolite or prodrug of any of these compounds, and combinations thereof.

67. The method of claim 52, wherein the subject is not administered quinidine.

68. The method of claim 52, wherein the subject is a human subject.

69. A method of treating a neurological disease or disorder in a subject in need thereof, the method comprising applying the transdermal patch of claim 20 to the subject.
